(19) United States
(12) Patent Application Publication
(10) Pub. No.: US 2019/0144178 A1
(43) Pub. Date: May 16, 2019

(54) MIXING CONTAINER AND METHOD OF USE

(71) Applicant: Cynthia S. McKenzie, (US)

(72) Inventors: Cynthia S. McKenzie, Reno, NV (US); Matthew T. Fisher, Reno, NV (US)

(73) Assignee: Cynthia S. McKenzie, Reno, NV (US)

(21) Appl. No.: 16/246,212

(22) Filed: Jan. 11, 2019

Related U.S. Application Data
(63) Continuation-in-part of application No. 15/788,656, filed on Oct. 19, 2017.
(60) Provisional application No. 62/410,063, filed on Oct. 19, 2016.
(60) Provisional application No. 62/550,185, filed on Aug. 25, 2017.

(51) Int. Cl. B65D 51/28; A45D 19/06; A45D 40/00; A45D 40/24; A61K 8/22; A61Q 5/06; A61Q 5/10; B65D 41/04; B65D 77/04; B65D 81/32

(52) U.S. Cl. CPC B65D 51/28; B65D 21/0209; A45D 40/0068; A45D 40/24; A61K 8/22; A61Q 5/065; A61Q 5/10; B65D 41/0435; B65D 77/04; B65D 81/32; A45D 2200/058; A45D 2200/25; A61K 2800/87; A61K 2800/882; A45D 19/06

(57) ABSTRACT

A custom hair coloring container, components of the container, and method of use and doing business. In one embodiment, the container includes a container body, a hair colorant container section of the container body removably mountable to a peroxide container section of the container body, and the hair colorant container section and the peroxide container section collectively forming a cavity with an inside surface. The hair colorant container section may include a first interior seal threadedly engaged with the inside surface within the hair colorant container, and the peroxide container section may include second interior seal threadedly engaged with the inside surface within the peroxide container section.

### RELATED APPLICATIONS

This Application is a Continuation-in-Part of U.S. patent application Ser. No. 15/788,656, filed Oct. 19, 2017, titled “HAIR COLORING KIT METHODS OF USE AND DOING BUSINESS,” which claims priority through the applicant's prior Provisional Patent Application Ser. No. 62/410,063, filed Oct. 19, 2016 titled “CUSTOM COLOR RETOUCH KIT AND METHOD,” and Provisional Patent Application Ser. No. 62/550,185, filed Aug. 25, 2017 titled “CUSTOM COLOR RETOUCH KIT AND METHOD.” All of such prior Applications are incorporated herein by reference in their entirety, but in the event of any inconsistency between either such prior Applications and this Application, this Application shall govern.

This specification contains material subject to copyright protection. The copyright owner has no objection to the photocopy reproduction of the specification and patent documents in exactly the form they appear in the Patent and Trademark Office patent file or records, but otherwise reserves all copyrights.

### FIELD

The present specification generally relates to the field hair color retouching and more particularly to one or more of a kit, and one or more components of a kit, for hair coloring, and methods of use and doing business to provide hair color retouching or one or more retouching components. The kit and its components can have other applications as well.

### SOME ASPECTS OF THE BACKGROUND

Hair coloring has long been a very large industry for a very long time. Today, it is an enormous industry in the U.S. alone.

For example, a First Research report states that, in 2014, there were 82,000 hair salons in the U.S. alone, in addition to 4,000 barber shops. A 2008 Clairol Survey reported that 75% of women in the U.S. between 18 and 65 years of age color their hair, and that 88% of women feel that their hair has an effect on their confidence. Multi-Sponsor Surveys reported in 2012 that 11% of men aged 50-64 color their hair.

A 2005 P&G Beauty Study done by Dr. Frauke Neuser showed that, of the women who color their hair, 52% do so at home only and 48% do so at a Salon (28% at a salon only and 20% at a salon and at home). In 2015, Modern Salon Media conducted a Process Haircolor Research Study, which found that the number one reason all women surveyed—home color consumers and salon clients combined—say they color their hair is to color gray and look younger (46%). For women 35 and older, gray coverage becomes even more important, at 65%. This same study found that 48% of salon color clients say they visit a salon every six weeks or more often for services, and 52% visit less frequently and that the overall average interval between salon visits is 7.5 weeks. This same survey found that most salon professionals (64%) say they recommend a “root cover-up” between color appointments, provided either professionally or by use of off-the-shelf coloring products, but 74% of color clients say they have either never had a root cover-up or are unsure if they have ever had one.

Since human hair generally grows at the rate of ½ inch per month, the enormous number of people who have hair coloring done professionally are experiencing substantial regrowth of uncolored hair between coloring services. For most people, the mismatch regrowth is quite visible in the front hairline and hair part within 2 to 3 weeks after a hair color treatment.

How Hair Coloring Works:

The visible part of the hair that is protruding from the skin is called the shaft. The structure of hair shaft is made up the three layers. The outer layer is called the cuticle and consists of overlapping dead cells (like roof shingles) which protect the inner layers. When the cells lie flat the hair reflects light and looks shiny. When the cells of the cuticle layer are open, the hair feels rough, dry, and looks dull.

The next layer is called the cortex. This is the layer that contains pigment, called melanin, that gives hair it's color.

The innermost layer is called the medulla. This layer is not involved in the color process.

There are three main compounds present in hair dye: ammonia; hydrogen peroxide, and dye intermediates and couplers. Hair typically has a pH level of 5. Ammonia, which is alkaline, is used to initiate the color process by swelling the hair shaft and opening up the cuticle layer.

Hydrogen peroxide is an acid that causes oxidation. The oxidation decolorizes (lightens) natural melanin pigment in the hair cortex and reacts with the color molecules of the chosen permanent hair color pigment to form dye molecules that cannot be washed out.

Hydrogen peroxide is added to hair dye in different concentrations. These concentrations are measured by how many volumes of oxygen are liberated from the decomposition of the hydrogen peroxide, meaning that 10 volume peroxide will form 10 volumes of oxygen from a single volume developer. The higher the concentration of developer, the more oxidation that will

occur in a client's hair.

Hydrogen peroxide thus initiates the color process and creates longer lasting color. The larger the volume of peroxide, however, the greater the amount of sulfur is removed from the hair. Loss of sulfur causes hair to harden and lose weight. This is why for most hair coloring services, the peroxide level is maintained at 30 volume or less.

The dye or coloring intermediates and couplers are small compounds that fit into the opened cuticle of the hair shaft. Once they mix with hydrogen peroxide, they become larger color molecules that cannot depart the hair shaft.

How Professional Permanent Hair Color is Used:

The professional hair colorist begins by examining the client's hair texture, dryness, natural hair color, and amount of gray hair, which is coloring resistant. From those factors the colorist determines the formulation of various pigments (dye) and enhancers to be used to achieve the desired result. Next, the colorist determines the volume of peroxide required according to the various factors. Then the colorist blends the pigment formulation with the peroxide formula. When the mixture is completely blended, it is applied to the client's hair.

The chemical process of pigment (color) becoming permanent in the hair shaft usually requires between 20-30 minutes to complete. The color formulation remains on the client's hair for that period of time in order to fully develop (replace color in the client's hair). After the prescribed period of time for development, the color molecules of the permanent hair color have expanded and are permanently fixed into the hair shaft. The hair is then shampooed, rinsed, and styled.

Because the peroxide component (H₂O₂, more fully referred to as “hydrogen peroxide”) is an acid, it is typically pre-mixed with water to dilute the peroxide in the resulting peroxide/water mixture. Nevertheless, even with this lower concentration of peroxide, if the resulting hair color formulation of colorant, water, and peroxide makes contact with human skin or hair for too long or with an excessive concentration of peroxide, the peroxide in the formulation can excessively oxidize, excessively dry, and damage the skin or hair or at least leave it with a dull finish or lack of shine. This is one reason why the professional colorist usually applies the colorant formulation with plastic gloves, to protect the colorist's skin from contact with the peroxide component of the formulation. This is also why the professional colorist makes adjustments to the colorant formulation applied to a given client in repeat coloration sessions over time, to ensure that the client's hair remains in a healthy condition. These risks are reasons that hair coloring is so commonly performed by a professional hair colorist.

Another reason is that, as noted above, the colorant component is typically customized by the professional colorist, by mixing one or more color components (usually at least two to three pigments or dyes), often with one or more other enhancers, to yield the color shade desired in the resulting colorant formulation. The professional hair colorist typically has in-depth knowledge of differing brands of hair coloring components and their differing qualities; and this knowledge plays a crucial role in adjusting the shades and strength of colors for differing clients, including by taking into account each client's hair texture, dryness, hair density, and natural color, including the amount of gray hair. After sufficient processing time in the client's hair, which can vary by client, the colorist rinses the client's hair to remove remaining colorant formulation not absorbed the hair. For all these reasons and others, such as convenience and combining the hair coloring process with procuring a haircut, clients seeking hair coloring commonly have the hair coloring formulation prepared and applied by professional hair colorists rather than by the clients on their own.

The Age-Old Problem of Hair Regrowth after Hair Coloring:

Within one to two weeks after hair is colored by professional colorists, clients typically experience visible naturally-colored hair regrowth, particularly around their front hairlines and at any parting lines within their hair. Since most hair color clients do not have the time or money to have their hair color professionally retouched every one to two weeks, their only options are to either maintain the visible regrowth, or to try and match the professional color with a hair coloring product acquired at a store or from an online commercial source.

The latter nonprofessional hair color products are, however, vastly inferior to professional hair coloring. One reasons is because over-the-counter products, designed as one size fits all, typically include an excessive concentration of peroxide—sometimes close to 40 volume in order to ensure maximum coloring of resistant gray hair—as compared to the much lower concentration that would be utilized by professional colorists for most clients.

In addition, since, as noted above, the professional colorist typically mixes different colors and possibly enhancer—often multiple different enhancers as well—to achieve a desired hair color shade for the client's particular type of hair and natural color, the client usually does not have the knowledge or formulation components to achieve anything close to the professional's hair color shade in the client's hair. The results of a non-professional use of off-the-shelf hair coloring products commonly are bands of colors that do not match, do not last and or easily washed out by shampooing, can cause the scalp to itch, and/or do not provide the desired resulting hair color, particularly in grey hair.

With regard to grey hair in particular, many off the shelf retouching products contain little or no peroxide. With little or no peroxide in the hair coloring formulation, grey hair cuticle in particular does not open and therefore does not become colored.

For these and other reasons, it is also common for clients who use home coloring products or kits to at least eventually experience an undesirable result in hair color and hair damage. When this occurs to a given client, the client's choices are to either live with the problem until the client's hair grows out, or have the problem corrected by a professional colorist. The corrective process is typically much longer and more expensive that normal professional coloring services, and the process usually yields yet further dryness and hair breakage.

So, while home-hair color products have long been widely available and themselves a substantial part of the huge hair coloring industry, they have long provided results that are inferior to the typically much more aesthetically attractive results of professional hair coloring, including when used to re-touch hair color between professional coloring sessions. At the same time, professional colorists have typically constantly sought additional ways to enhance their product and service offerings and increase resulting profitability as well. This has been the state of the hair coloring industry for well over 40 years.

### BRIEF SUMMARY OF SOME ASPECTS OF THE SPECIFICATION

The applicant has developed a hair coloring kit and method of use and doing business. In some embodiments, the kit provides pre-mixed colorant (such as multiple differing hair coloring colors and, optionally, one or more hair coloring enhancers), and optionally peroxide, to or for an individual, with the colorant custom pre-mixed to match or compliment other hair colorant applied to the individual. The individual or other person can then mix the pre-mixed colorant with the optional or other peroxide for application of the resulting colorant formulation to hair on the individual.

In some methods, the pre-mixed colorant is pre-mixed by or for a professional hair colorist, and some methods include the pre-mixed colorant being provided by or for the professional hair colorist to or for a client of the professional hair colorist. Some methods include generation of the pre-mixed colorant during or in connection with a hair coloring session for the client. In some instances, the pre-mixed colorant is a portion of colorant also generated and applied to the client during a professional hair coloring session.

In some embodiments, the kit and method can further be used to solve the age-old problem of maintaining the color of hair, such as regrowth for example, after a hair coloring session, such as a professional hair coloring session for example. Consequently, some instances can include the kit being used by or for the client to color the client's hair after the hair coloring session, which may take place, in some embodiments, up to weeks, months, or more after the hair coloring session.

The kit can include, in addition to pre-mixed colorant and/or peroxide, a mixing container, a mixing tool, an applicator brush, instructions for use, one or more other possible components such as a mirror, which may be lighted, and packaging for the kit. The packaging or one or more other components may have indicia of varying types, and in some embodiments some or all the indicia may be customized for the hair colorist professional, a salon, or provider of the kit. In some kits, the packaging or other one or more other components can include outer trademark indicia and may also include indicia otherwise customized, such as by printing or marking on the actual packaging or by label(s) applied to the packaging.

Packaging for some kits can have an attractive shape, such as a triangular outer shape for example. Some instances can provide a relatively small, lightweight, or easily displayed package.

One particular type of packaging can have a plurality of triangular sides and be small, lightweight, and easily displayed on either a shelf or by being hung on a mounting rod or other mounting element. The packaging may include a mounting rod channel passing through the packaging—for example, through a narrower end of the packaging as compared to an opposed thicker end.

Some kits may include a single container providing separate chambers or compartments for storage of colorant separately from other material such as peroxide for example. The colorant may then be mixed with the other material by opening the container, removing one or more sealing structures, and mixing the colorant with the other material.

Some instances of the container can include a first chamber and a second chamber, and the first and second chambers may be removably mountable to each other such as by mating interlockable threads for example. The first chamber may have a first removable external seal, such as on the one outer end of the first chamber and may have a second or other seal removable mountable within the first chamber spaced from the first removable seal. The second chamber may include another chamber seal as well.

The packaging and container may be sized so that separate container compartments can be secured within the container and, when the packaging is opened, the separate compartments can be filled with differing hair coloring materials, removed from the packaging and mounted together to provide the container and mixing of the differing hair coloring materials within the container, and then remounted within the packaging along with any other kit components. In some embodiments, the packaging may have a plurality of triangular sides providing a relatively thickened end opposite a pointed end, and the mixing container can be mounted in the thickened end of the packaging for delivery to or for a hair coloring client.

Some methods of mixing container use may include placing hair colorant in the first or second chamber of the container and peroxide or other material in the other chamber of the container. Some such methods may include then inserting a second or other removable seal in the first chamber and, in some instances, the second chamber removable seal in the second chamber. The first and second portions may then be mounted to provide a container containing the colorant and peroxide or other material, with at least one removable seal separating the colorant from the peroxide or other material within the container.

The colorant and peroxide or other materials may then be mixed by separating the first section of the container from the second section, removing at least one or more removable seal, remounting the first and second sections to provide a container and mixing of the colorant, peroxide, or other materials within the container.

Some embodiments of the container provide the first chamber of the container having both the first removable seal and second removable seal spaced from the first removable seal and optionally a second chamber removable seal within the second chamber. The differing components are placed in the differing container sections so that they are maintained separately from each other by the removable seals. The components may then be mixed by separating the container chambers, removing one or more seals, remounting the container chambers to each other, removing an outer seal to provide an open passage into the container, such as in an upper end in the container, and mixing the materials within the container such as with a mixing tool penetrating the open passage.

In some embodiments, one or more of the hair colorant or other coloring process material may be in a gel within the container. The gel may be sufficiently thick and self-adhering that the gel remains in place within the container even after associated sealing structure is removed. In some such embodiments, the container section containing the gel can therefore be separated from another container section to remove the associated sealing structure and then remount gel containing section over the other container section without the gel spilling out of the gel containing section. Some instances can include an upper removable seal that is then removable in order to mix the components within the container such as with a mixing tool.

In some embodiments, the mixed coloring components in the container can thus provide colorant that can be applied to hair. The hair may be that of professional colorist client in order to maintain the desired color on the client after receiving professional hair coloring by or for the professional colorist.

Some instances of the method can thus provide a promotional or revenue source, such as for a salon, a professional hair treatment center or colorist, or other business or activity. The kit can be sold for example. It may also provide a marketing or promotion vehicle, including in some instances through inclusion of indicia on one or more of the kit packaging and components.

Alternative embodiments of a mixing container can have a removeable seal that is threadably mountable within the first or second chamber. Some applications can have at least two such seals mountable within either the first and second container chambers. In some embodiments, such seals can more securely seal matter within the differing container chambers and then be unthreaded for each of their respective container chambers to gain access to the interior of each such chamber and, for example, insert matter within them.

In some instances, one or both removeable seals can have opposed radially outwardly extending flanges that can abut mating structures on a container chamber so that as first and second container chambers are threadably or otherwise interconnected, the one or more flanges provide sealing contact with the mating container structure. In the case of two seals each having such flanges, in some embodiments they can forcefully abut each other by such interconnection as well. Later, when desired, the first and second container chambers can be separated, allowing the seals to be removed, such as by, in some embodiments, merely unthreading them from their respective container sections. Some applications can thus provide a mixing container in which differing compositions can be placed differing container chambers and securely sealed in the container chambers respectively while the differing container chambers are threadably mounted to each other to provide a single container containing the differing compositions separate from each other. The mixing container can then be transported, stored, or later opened without intermixing or leaking of the differing compositions. The first and second container chambers can then be readily separated and the removable seals can then be removed from their respective container sections, so that one composition can then be mixed with other as desired; and in some applications optionally the two container chambers can again be sealingly threadably mounted to each other, with or without the first or second seals, for transport, storing, or disposing of the mixing container and any remaining contents.

Some embodiments of the mixing container and its components can be made of lightweight yet durable material. For example, the container sections can be made of plastic, and first and, if present, second seals can be made of rubber or flexible but resilient plastic. When present, the external seal of the first container section can be made of a flexible sealing foil or plastic.

It is to be understood that, while much of the foregoing discussion has been dominantly in the context of human hair coloring, the hair involved may be animal or plant hair or any other regrowth of any type. Further, the kit components may be mixed and matched as desired and may be used in applications and with components other than those identified in this Brief Summary section.

It is also to be understood that there are other novel aspects of this specification. They will become apparent as this specification proceeds. In this regard, the scope of the invention is to be determined by the claims as issued and not by reason of whether a given feature or feature set is included in this Brief Summary or addresses an issue in the Background section of this specification.

### BRIEF DESCRIPTION OF THE DRAWINGS

The applicant's preferred and other embodiments are shown in the accompanying drawings in which:

FIG. 1 is a perspective view of a hair coloring kit of this specification;

FIG. 2 is a perspective view of another hair coloring kit of this specification opened and showing contents of the kit and having a right-angled corner in opposed triangular sides of the package;

FIG. 3 is perspective view of hair coloring mixing container of the kit of FIG. 2;

FIG. 4 is a right-side elevational view of the hair coloring mixing container of FIG. 3;

FIG. 5 is a top plan view of the hair coloring container of FIG. 3;

FIG. 6 is an exploded perspective view of the mixing container of FIG. 3;

FIG. 7 is a cross-sectional view taken along section line 7-7 of FIG. 5;

FIG. 8A is a cross-sectional view taken along section line 8A-8A of FIG. 2;

FIG. 8B is a perspective view of the container top section of FIG. 9A and mixing spatula and hair coloring gel during insertion of the gel into the top section;

FIG. 8C is a perspective view of the container bottom section of FIG. 9A with peroxide poured into the bottom compartment;

FIG. 8D is a perspective view of the container top section of FIG. 8A and a removable top section resilient seal prior to insertion into the interior of the container;

FIG. 8E is a perspective view of the container bottom section of FIG. 9A and a removable bottom compartment resilient seal prior to insertion into the interior of the container bottom compartment;

FIG. 8F is a perspective view of the container top section being mounted to the bottom section (conversely by reverse rotation with respect to each other, being separated from the bottom section);

FIG. 8G is a perspective view of the assembled hair coloring container of FIG. 9E;

FIG. 8H is cross-sectional view taken along section 8H-8H of FIG. 9G;

FIG. 8I is perspective view of the assembled hair coloring container placed in the open kit packaging;

FIG. 8J is a perspective view of the assemble hair coloring container of FIG. 9G but with the top compartment's top side flexible seal being removed;

FIG. 8K is a perspective view of the hair coloring container of FIG. 8H but with the top compartment's top side seal removed and the kit's spatula penetrating the exposed channel in the top side of the top compartment;

FIG. 8L is a perspective of the hair coloring container of FIG. 9K but with the kit's applicator brush penetrating the exposed channel to pick up mixed hair colorant with peroxide;

FIG. 9 is a perspective view of the kit package of FIG. 2 with its extended lip sealed around the upper end of the package in the orientation of FIG. 9, and showing the ornamental external configuration of the kit package;

FIG. 10 is front elevational view of the kit package of FIG. 9;

FIG. 11 is a rear elevational view of the kit package of FIG. 9;

FIG. 12 is left side elevational view of the kit package of FIG. 9;

FIG. 13 is a right-side elevational view of the kit package of FIG. 9;

FIG. 14 is a top plan view of the kit package of FIG. 9;

FIG. 15 is a bottom plan view of the kit package of FIG. 9;

FIG. 16A is a perspective view of an alternative embodiment of a hair coloring mixing container;

FIG. 16B is a cross-sectional view of the container of FIG. 16A taken along section line A-A of FIG. 16A;

FIG. 17 is a perspective view of a hair colorant section of the container of FIG. 16A;

FIG. 18 is a bottom plan view of a hair colorant section of the container of FIG. 16A;

FIG. 19 is a side plan view of a hair colorant section of the container of FIG. 16A;

FIG. 20 is a top perspective view of a hair colorant section of the container of FIG. 16A;

FIG. 21 is a bottom perspective view of a hair colorant section of the container of FIG. 16A;

FIG. 22 is a top perspective view of an interior seal;

FIG. 23 is a bottom perspective view of an interior seal of FIG. 22;

FIG. 24 is a side plan view of an interior seal of FIG. 22;

FIG. 25 is a top perspective view of a peroxide section of the container of FIG. 16A;

FIG. 26 is a side plan view of the peroxide section of the container of FIG. 25;

FIG. 27 is a perspective view of the peroxide section of the container of FIG. 25; and

FIG. 28 is a block diagram of a method of using a container.

### DETAILED DESCRIPTION

The following description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

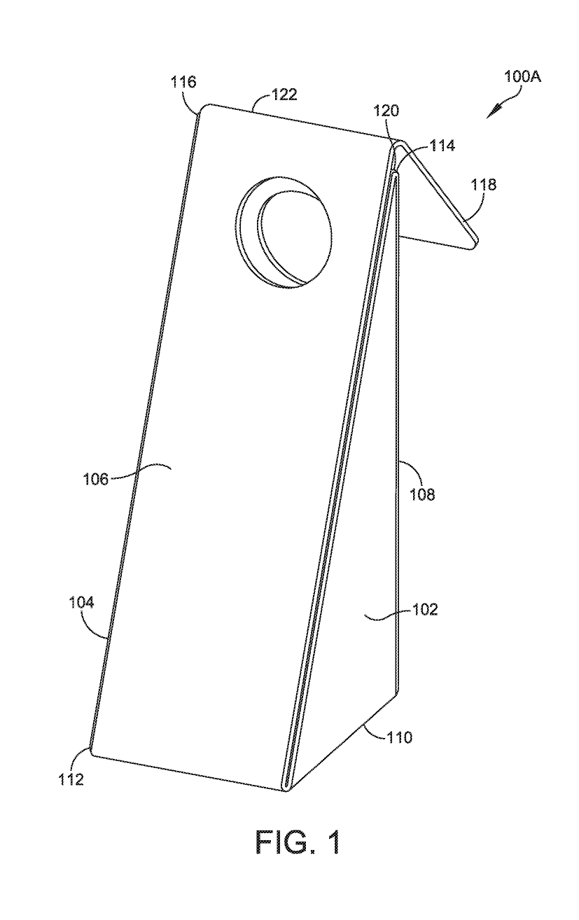

With reference now to FIG. 1, one embodiment of a hair retouching kit package, generally 100A, has two opposed, parallel, planar triangular sides 102, 104 spaced apart from each other by two mostly opposed and mostly planar rectangular sides 106, 108. Each rectangular side, e.g., 106 extends from the widened ends 110, 112, of the opposed triangular sides 102, 104, respectively, to the narrower pointed ends 114, 116, of the opposed triangular sides 102, 104, respectively. One rectangular side 106, however, also has an extended folding end 118 that can fold around the opposed rectangular side end 120 spanning between the opposed narrower pointed ends 114, 116 of the opposed triangular sides 102, 104. A circular mounting pole passage 122 extends through the opposed rectangular sides 102, 104 and through the extended folding end 118 spaced from, but relatively close to, the folding end 124 of the extended folding end 118.

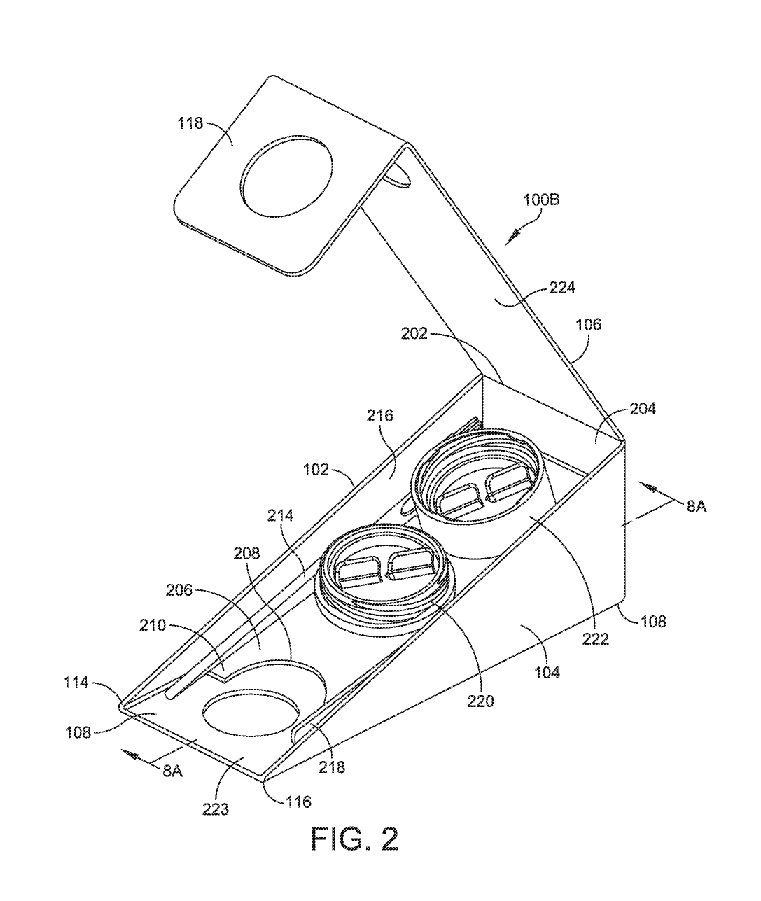

With reference now to FIG. 2, the kit package 100B can be opened by unfolding the extended folded end 118 and lifting that end 118 to cause the extended rectangular side 106 to rotate away from the pointed ends 114, 116 of the opposed triangular sides 102, 104 about the edge junction 202 of the widened package end 204 opposite the pointed ends 114, 116. A removable kit component mounting platform insert 206 extends from the widened end 204, spaced from the shorter rectangular side 108 opposite the extended rectangular side 106, toward the opposed pointed ends 114, 116 of the opposed triangular sides 102, 104 to terminate in a semi-circular cutout end 208 with opposed arms 210, 212 (212 not shown) abutting the shorter rectangular side 108 spaced from the opposed pointed ends 114, 116 of the opposed triangular sides 102, 104.

With reference now to FIG. 2, in another particular embodiment of the kit package 100BB: the triangle sides, e.g., 104, each have outer peripheral triangle dimensions of 3×8.25×8.75 inches. The widened end 204 is a 3×3 inch square. The rectangular lower side 108 has outer peripheral dimensions of 3×8.25 inches. These dimensions can be varied by plus or minus 60%. The package 100B and its removable mounting platform insert 206 can each be made from a single folded piece of paper, cardboard, plastic sheet, or other suitable material.

Multiple kit packages, e.g., 100B, are thus readily stackable and packaged, such as by inverting one package with respect to another and rotating it 180 degrees in a vertical plane and stacking the inverted package on top of a base non-inverted package to yield a rectangular box-like structure provided by the stacked two packages. This same stacking technique can be repeated as many times as desired, including if desired by placing stacked packages side-by-side, to yield and overall rectangular structure of packages, e.g., 100B, without any wasted or unused space in the stacked package structure.

The kit package 100B contains an applicator brush 214 abutting the interior side 216 of one triangular side wall 104, a mixing spatula 218 abutting the opposing interior side (not shown) of the opposite triangular side wall 102, a mixing container top compartment 220 mounted inverted in this location, and the bottom compartment 222 mounted upright intermediate the applicator brush 214 and mixing spatula 218. The kit package may include kit use instructions (not shown), and they may be folded and contained under the component mounting platform insert 206 or printed on the interior side 224 of the extended folded end 118.

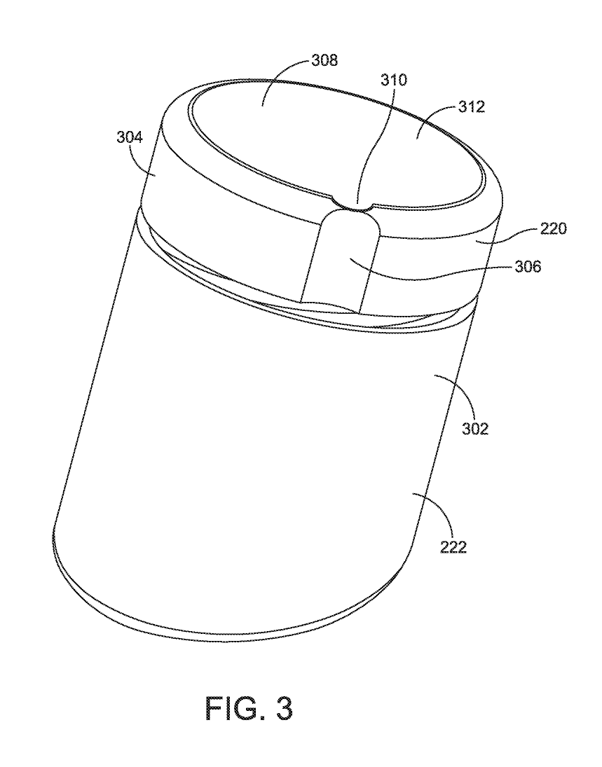

With reference now to FIG. 3, the assembled mixing container 302 has the generally tubular top compartment 220 removably threaded to the relatively larger, generally tubular bottom compartment 222. The outer circumferential periphery 304 of the top compartment 220 has a finger placement indent 306 penetrating radially inwardly through the circumferential periphery 304. The planar top side 308 of the top compartment 220 consists a removable, generally circular, removable planar seal 308 spanning across a tubular channel (not shown in FIG. 3) in top compartment 220 underlying the circular planar seal 308. The planar seal 308 has a grippable lip 310 extending radially outwardly from the central circular body 312 of the circular planar seal 308.

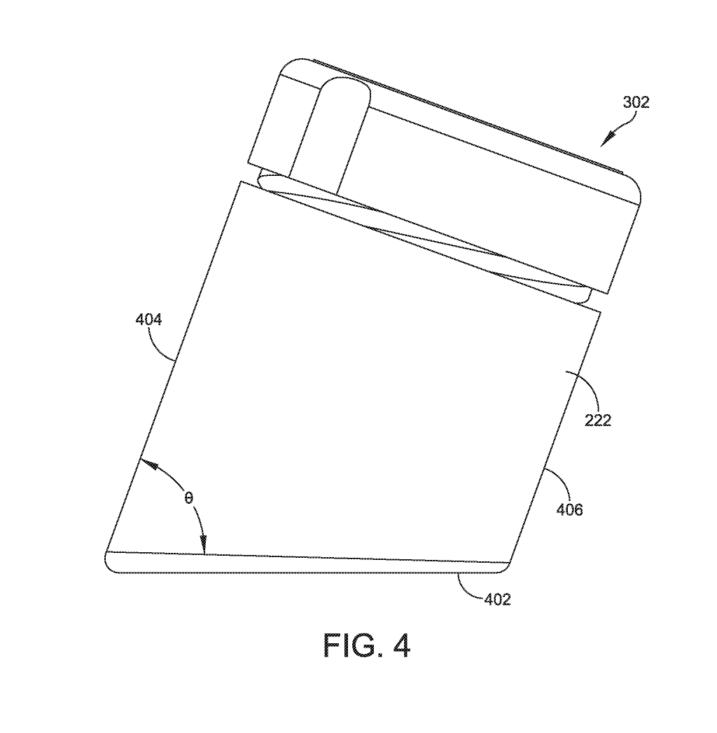

With reference now to FIG. 4, the bottom compartment 222 has a planar bottom end 402 extending at an acute angle from the longer laterally extending side 404 of the bottom compartment 222 to the other, shorter laterally extending side 406 of the bottom compartment 222. In the depicted embodiment, that angle Θ is 73 degrees, bit this angle can be varied, such as up to plus 5 more degrees or less 25 degrees as but one example, to accommodate differently sized components or even those identified in detail in this specification. 73 degrees allows, as shown in FIG. 2, a larger volume bottom compartment 222 to be mounted in the kit package 100B with the planar bottom end (not shown in FIG. 2) parallel to, and matingly abutting, the adjacent portion (not shown in FIG. 2) of the planar interior side 223 of the shorter rectangular side 108 of the kit package 100B.

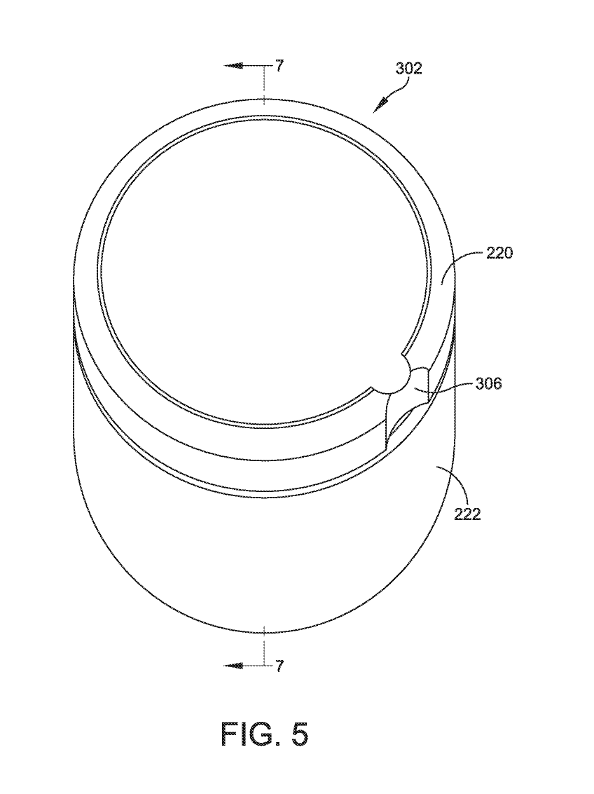

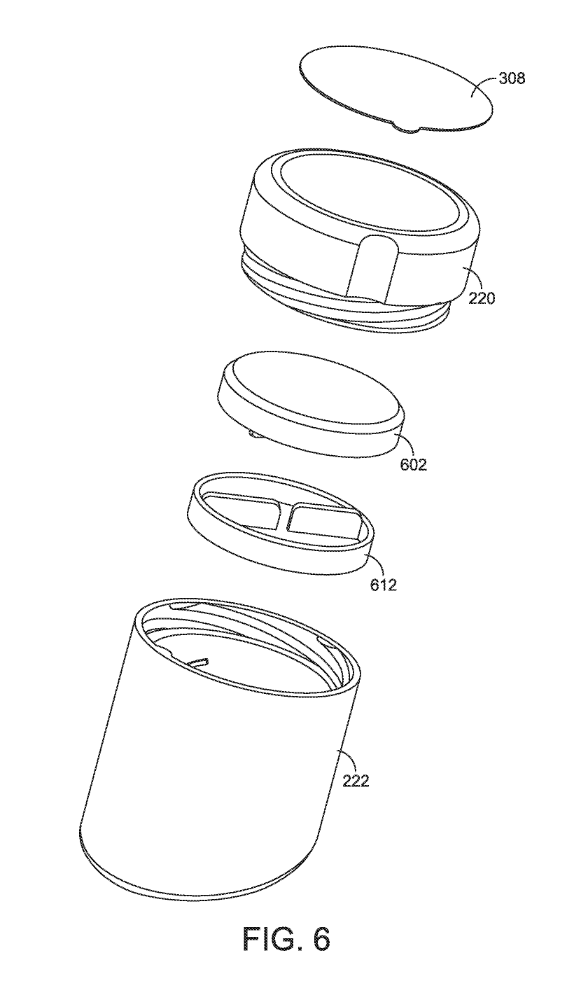

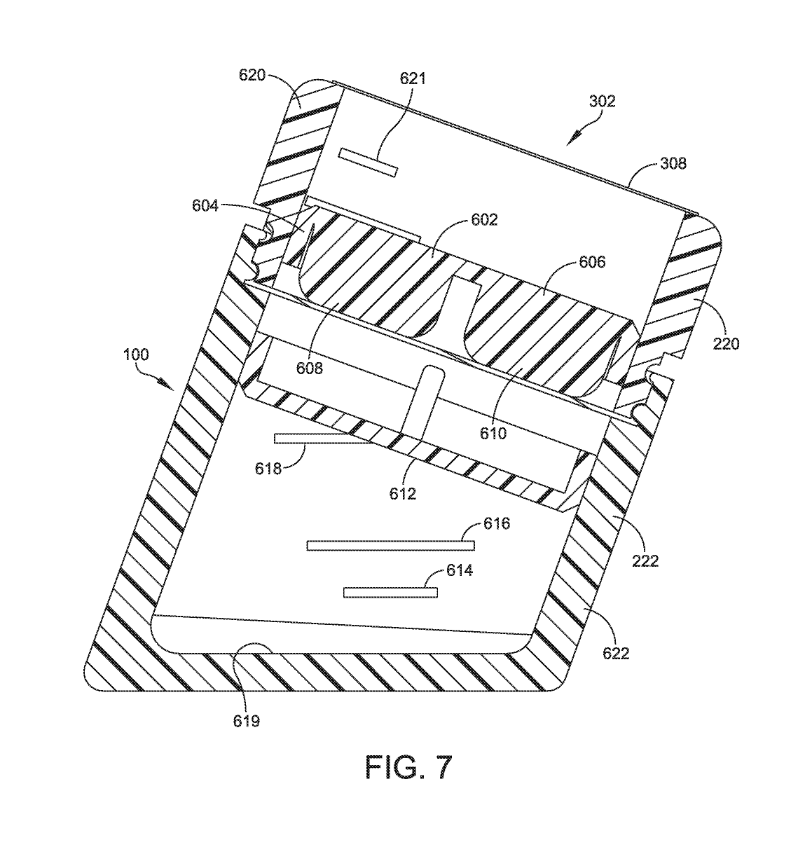

With reference now to FIG. 5, this top plan view shows how the assembled mixing container 302 also inherently tilts with respect a planar horizontal surface (not shown in FIG. 7), such as a table, on which the assembled mixing container rests. In this position, a person can grasp the top compartment 220 with one hand (not shown) and the bottom compartment 222 with another hand (not shown), while the thumb of the one hand penetrates the finger placement indent 306 in order to rotate the top compartment with respect to the bottom compartment.

Referring now to FIG. 6, the components of the mixing container 302 include not only the top compartment 220, its removable top planar seal 308, and the bottom compartment 222, but also a resilient, removable, and replaceable top compartment interior seal 602, and an identically structured but inverted (as compared to the top compartment interior seal 602) bottom compartment interior seal 612. The top compartment interior seal 602 is slidably mounted within the top compartment 220, and the bottom compartment interior seal 612 is slidably mounted within the bottom compartment 222. The components of the mixing container 302 may be made from materials as follows:
the top section 220 and bottom section 222 may be made of any suitable strong plastic (such as nylon, acrylic, PVC, polyethylene, polypropylene, polycarbonate, bakelite, melamine, etc.), glass, composite, or metal;
the interior seals 602 and 612 may be made of resilient material such as plastic for example; exemplary plastics can include polyethylene, polypropylene, polyurethane, etc.; and
the top seal 308 may be made of resilient material such as plastic (such as identified above), Teflon sheet, foil-lined paper, PET, aluminum sheet; and it 308 may be adhered in position on the top edge of the top section 220 by any suitable adhesive.

Referring now to FIG. 7, the mixing container 302 as delivered to a user, such as professional hair colorist for example, may be assembled as shown in FIG. 4 rather that in the separated format of FIG. 2. In either event, as provided for use by a professional colorist to provide a coloring kit to a client, the top interior seal 602 has (i) a tubular side sealing wall 604 extending from a planar sealing end 606 and (ii) opposed grippable tabs 608, 610 (a) extending downwardly from the planar sealing end 606 in the orientation of the seal 602 shown in FIG. 2 and (b) surrounded by the tubular side sealing wall 604. The opposed tabs 608, 610 are spaced from each other about the axis of the top compartment 220.

In addition, the bottom compartment seal 612 is spun about its axis, such as by 20 to 90 degrees, to the orientation of the top compartment inner seal 602. The spacing apart of the opposed tabs, e.g., 608, 610, allows the opposed interior seals 602, 612 to come closer together within the mixing container 302 if desired without the opposed tabs from the opposed seals 602, 612 bumping into each other in blocking fashion.

Each compartment has internal sidewall linear markings, e.g., 614, 616, 618. These markings provide fill level indicia to the user when the user places fluid or gel within each of the respective top and bottom compartments 220, 220 as explained further infra. The linear markings 614, 616, 618 of the bottom compartment 222 are parallel to the generally planar bottom surface 619 in the bottom end 402 of the bottom compartment 222. This allows the markings, e.g., 614, 616, 618, to be horizontal as the bottom compartment is filled with fluid as explained infra.

In contrast, the linear level markings, e.g., 621, of the upper compartment are parallel to planar upper seal 308 in the top compartment 220. This allows these markings, e.g., 621, to be horizontal as the top compartment 220 is inverted and filled with material as also explained further infra.

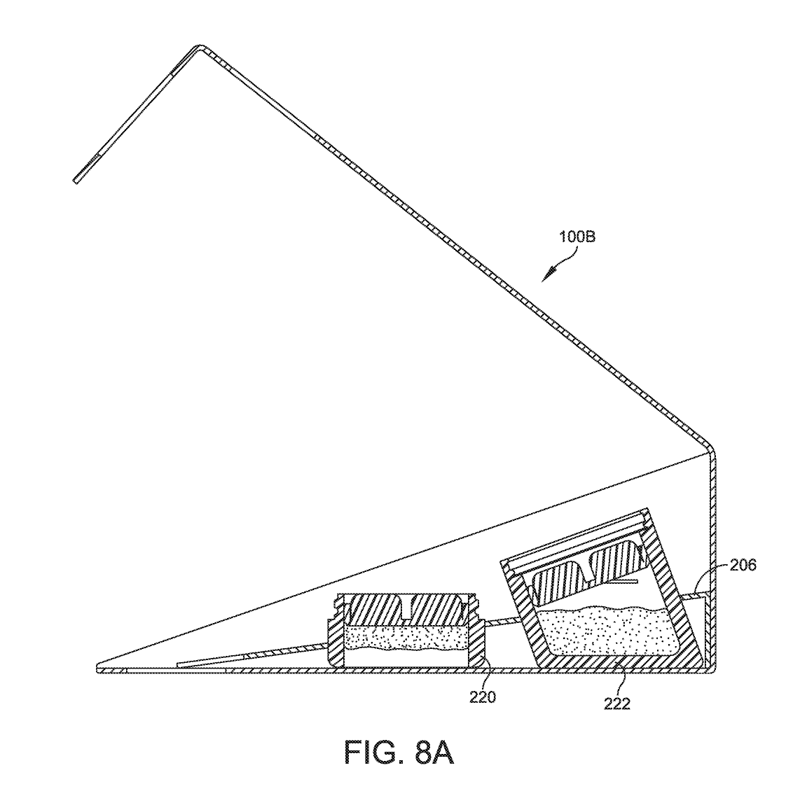

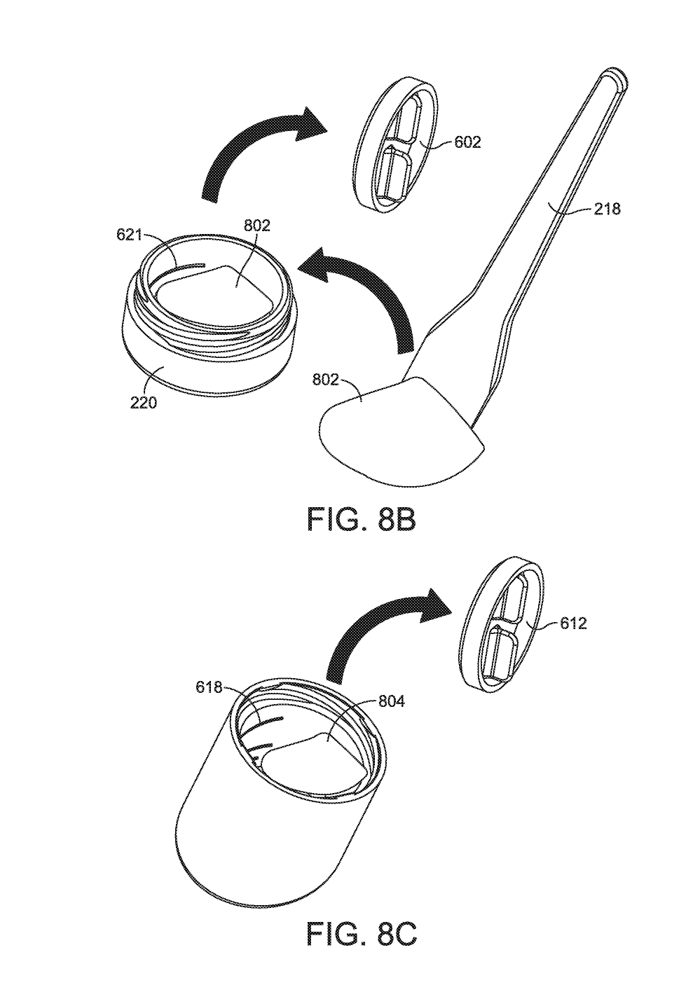

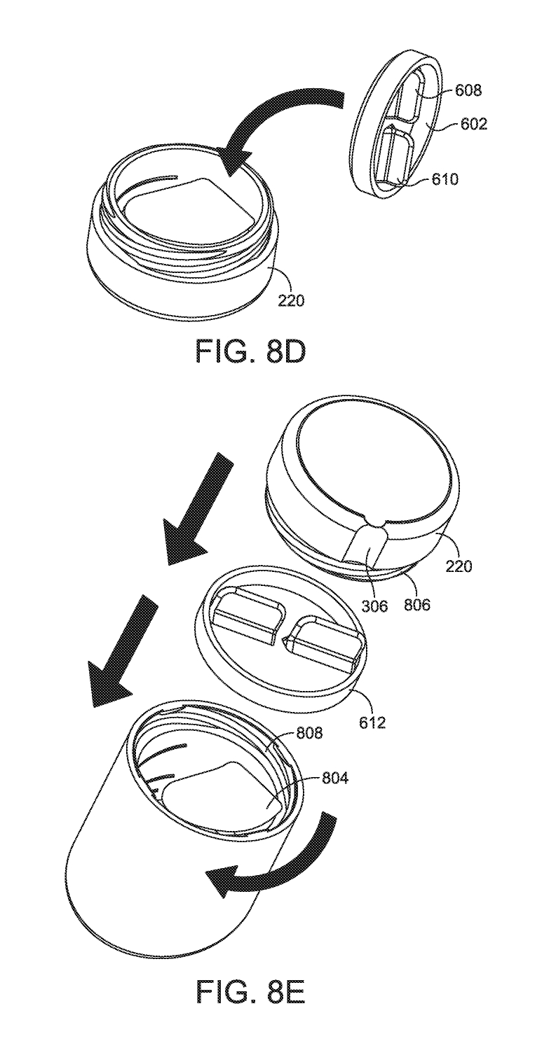

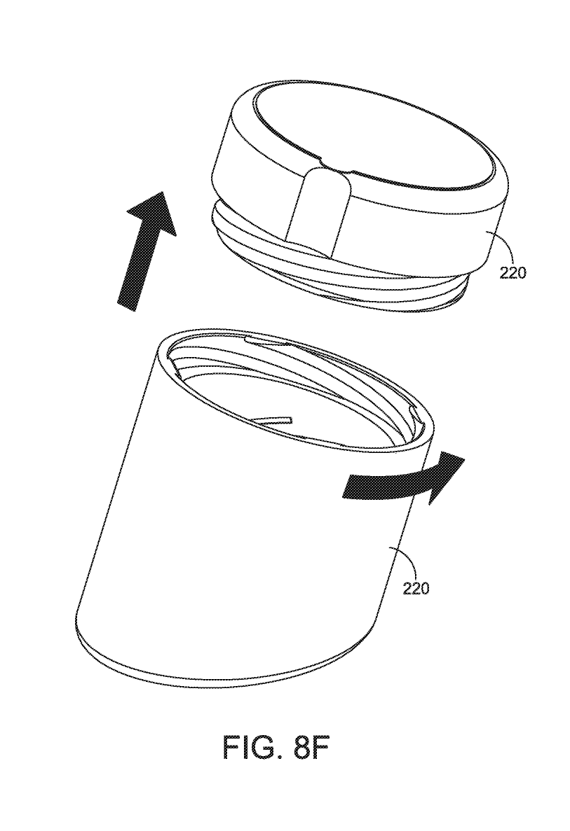

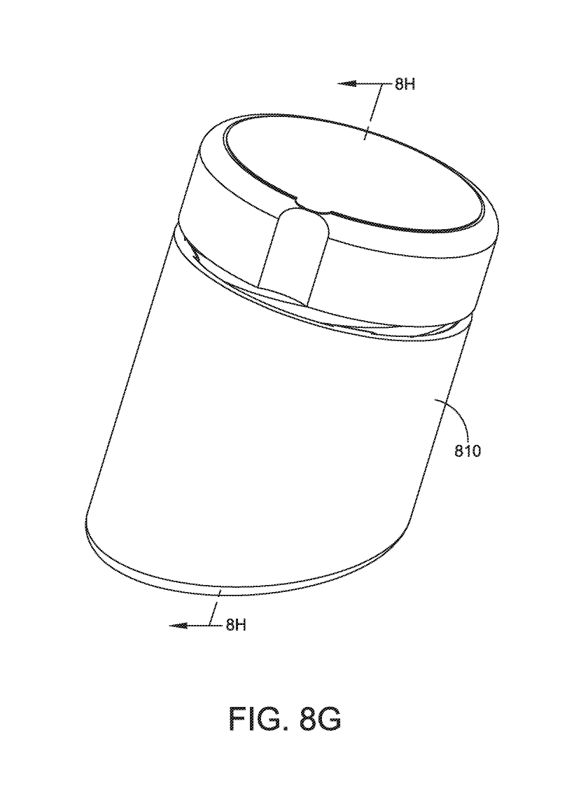

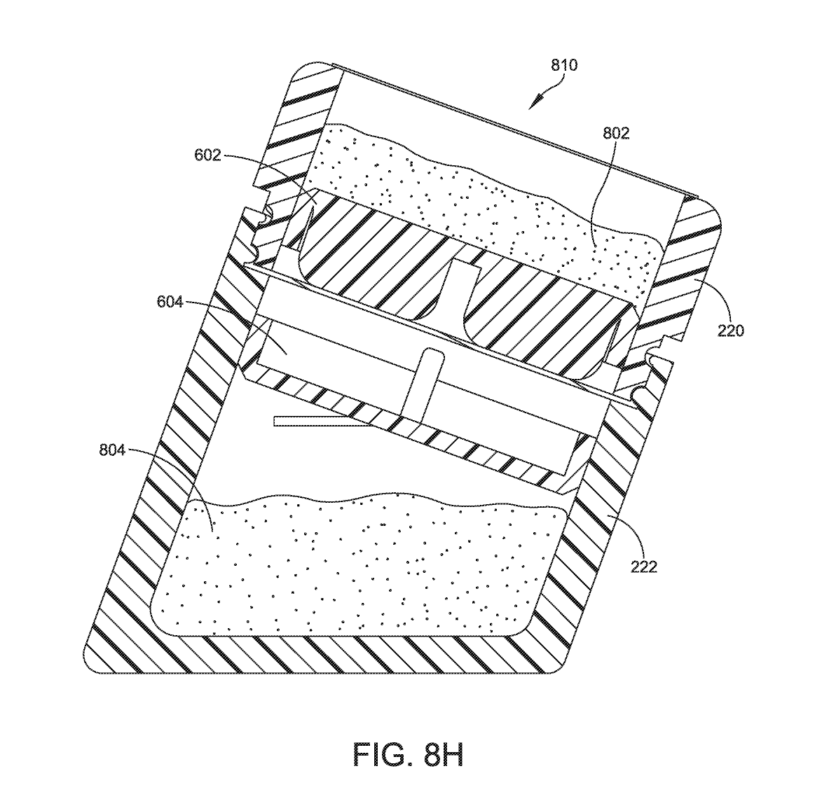

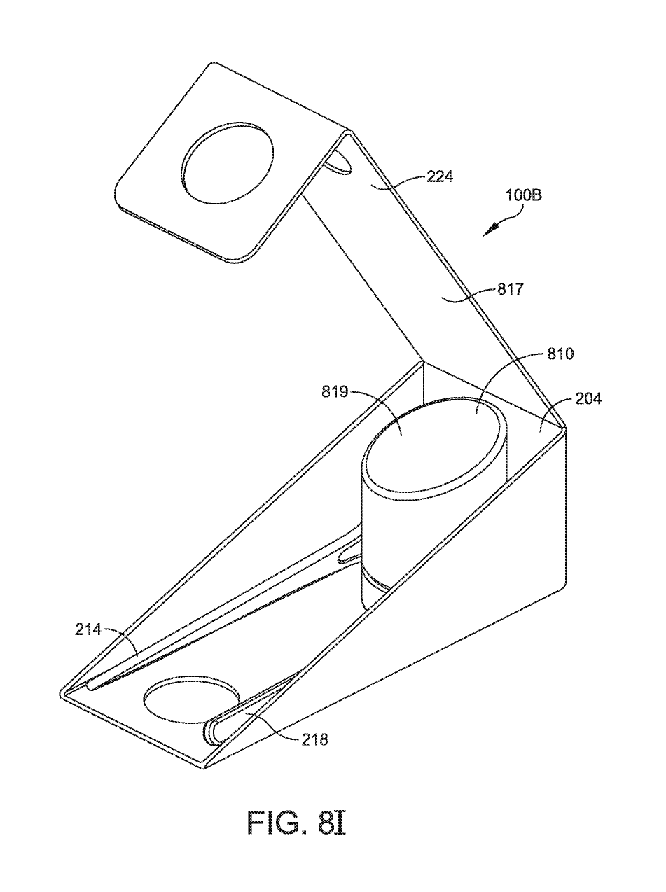

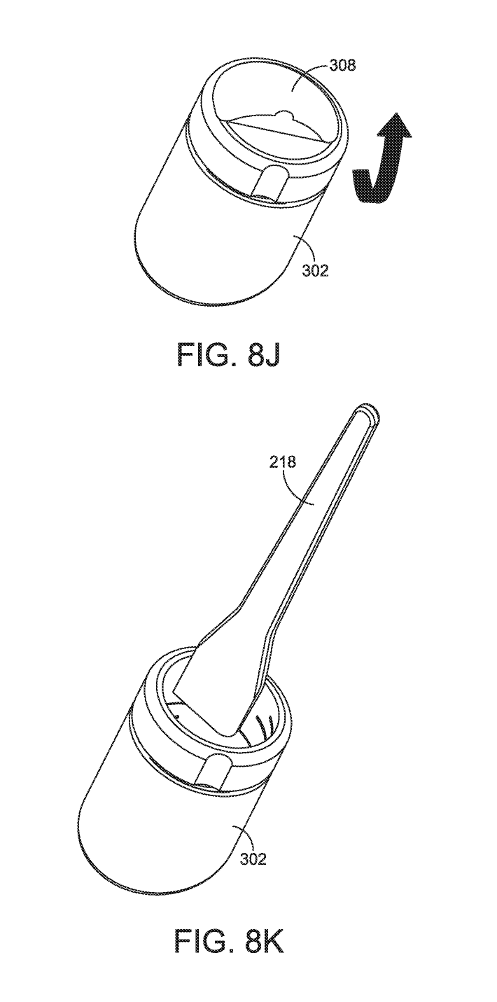

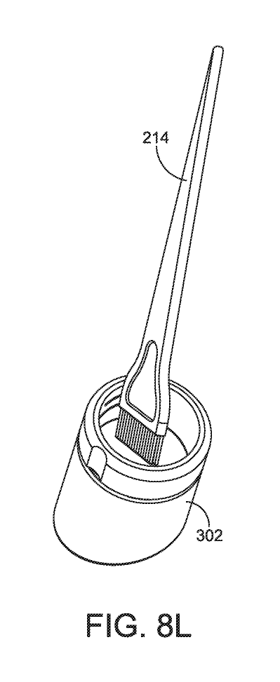

With reference back to FIG. 2, when the kit package 100B of FIG. 2 is to be used by or for professional colorist for a client, the colorist can:
prepare custom coloring formation for a client, commonly in connection with a hair coloring treatment provided by the colorist for the client; the formulation is made by mixing one or more differing colors and/or one or more differing enhancers and results in a relatively thick gel;
open the kit package 100B as shown in FIG. 2 and either remove the inverted top compartment 220, bottom compartment 222, and mixing spatula 214 from the kit package 100B, or just remove the spatula from the kit package 100B and leave the top compartment 220 and bottom compartment 222 in position in the kit package 100B and in the following fill steps, fill the top 220 and bottom 222 compartments in that position (see also FIG. 8A);
with reference to FIG. 8B, grasp the tabs of the top compartment interior seal 602 and remove the seal 602 from the top compartment 220, and use a spatula 802 to place the gel colorant 802 into the interior of the top compartment 220; in doing so, the colorist can use the top compartment linear markings, e.g., 621, to insert the proper quantity of coloring formulation to place within the top compartment 220 (0.5 ounce typically will be enough to treat at least the client's part line and front hairline regrowth after about two weeks; 1.5 ounce will typically be sufficient to treat all hair regrowth on the client);
with reference to FIG. 8C, grasp the tabs of the bottom compartment interior seal 612 and remove the seal 612 from the bottom compartment 222, and pour peroxide 804 (typically between 0.5 to 1 ounce), which the colorist may have previously customized for the client as well, into the interior of the bottom compartment 222; in doing so, the colorist can use the bottom compartment linear markings, e.g., 618, to determine the proper quantity of peroxide to place within the bottom compartment 220;
with reference to FIG. 8D, grasp the tabs of the top compartment's interior seal 602 to slide the interior seal 602 to abut the gel colorant 802 within the interior of the top compartment 220;
with reference to FIG. 8E, grasp the tabs of the bottom compartment's interior seal to slide the interior seal 612 to abut the upper surface of the peroxide 804 within the interior of the bottom compartment 222;
with reference to FIG. 8G then invert the top compartment 220 and, grasping the top compartment 220 with one hand and the bottom compartment 220 with the other, push against the finger indent 306 to thread top compartment threads 806 into mating bottom compartment threads 808 and lock the top compartment 220 to the bottom compartment 222 by friction fit between the opposed threads 806, 808, yielding the sealed container 810 as shown in FIGS. 8G and 8H;
as shown in FIG. 8I, remove the removable component mounting platform insert 206 (as shown in FIG. 8A), place the applicator brush 214 and mixing spatula 218 back in the kit package 100B, place the mixing container 810 in the package with the longest peripheral side 811 of the container 810 abutting the widened end 204, close and re-seal the kit package 100B (as shown in FIG. 9), and give the package and the sealed container 810 to the client or to someone for the client; note that in this orientation of the container 810 in the closed package 100B, the container 810 is generally secured in position by the opposed applicator brush 214 and spatula 218 abutting opposed sides 813, 815 of the container 810, the widened end 204 of the package, and the matingly slanted underside 817 abutting the slanted end 819 of the container 810; and, if desired:
repeat the above process to the extent desired to provide the client with one or more addition additional sealed colorant and peroxide containers, re-sealed kit packages, or other kit components as desired. Since each such container will typically be for a single hair coloring retouching service, providing one or more additional such containers can allow the client to have one or more additional retouching service sessions.

Differing entities may perform differing steps, or portions of steps above. In some embodiments, for example, the main objective can be to have the steps performed so that a custom hair coloring formulation container is provided to the professional colorist's client for later use by or for the client. Thus, various parts of the method, and components, also may not be utilized if desired.

Further, the kit may include yet other or multiple components. For example, the colorist may provide the client with multiple mixing containers so that the client may utilize one container for one retouching session and another container for a second retouching session.

The client or other user may later implement the following method, such as, for example, when hair regrowth occurs on the client:
with reference to FIG. 8I, open the kit package 100B and remove the mixing container 810, applicator brush 214, mixing spatula 218, and instructions if in the kit package 100B;
read the instructions, which as noted supra can be on the underside 224 of the package;
with reference to FIG. 8F, unscrew the mixing container's top compartment 220 from the bottom compartment 222 and remove the interior seals 602 and 612 from the top 220 and bottom 222 compartments, respectively (making sure to maintain the bottom compartment in an upright position);
the top container can be screwed to the bottom container to create a larger container for mixing the color formula with the peroxide;
with reference to FIG. 8J, remove the top side seal 308 from the mixing container 302;
with reference to FIG. 8K, use the mixing spatula 218 to mix the hair colorant with the peroxide in the container;
with reference to FIG. 8L, use the applicator brush 214 to dip into the mixed colorant-and-peroxide formulation to apply the mixture to regrowth hair; and
at the appropriate time rinse the mixture from the hair.

Again, differing entities may perform differing steps, or portions of steps, above. In some embodiments, for example, the main objective can be to have the steps performed so that at least custom hair coloring formulation is provided to the professional colorist's client for later use by or for the client. Various parts of the method, and components, may not be utilized if desired.

In one embodiment, the sealing container 302 may weigh from 0.1 to 0.5 lb. depending on its compositing and size. The entire package kit 100B such as shown in FIG. 2 can weigh from 0.3 to 1 lbs. These container and kit can thus be very lightweight.

Further, all components of the particular kit 100B shown in FIG. 2 can be recyclable, and the kit 100B is easy to package, store, and ship. If desired, the client can return the mixing container to the professional colorist for refilling of the mixing container or reuse of the mixing container by or for the professional colorist and/or the client or another person or entity.

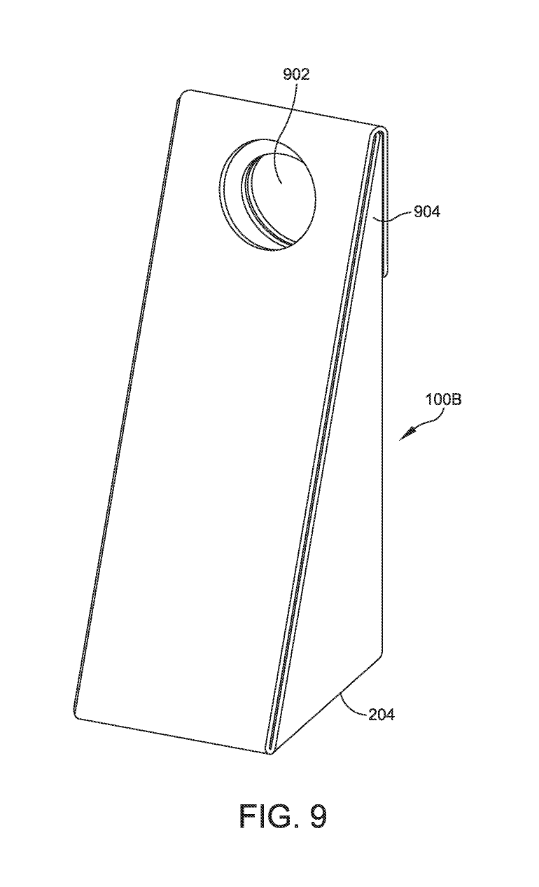

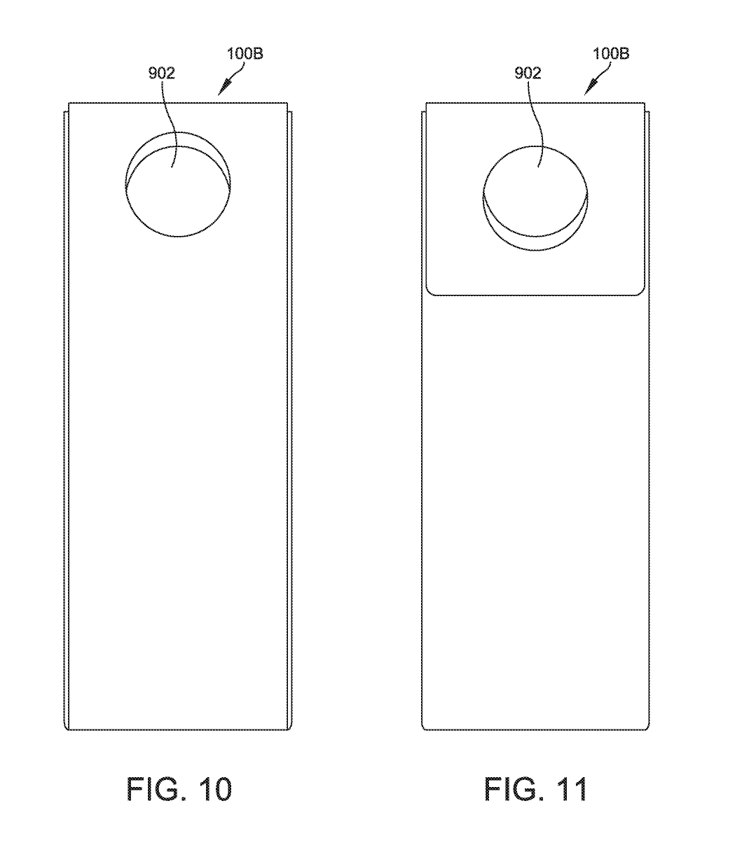

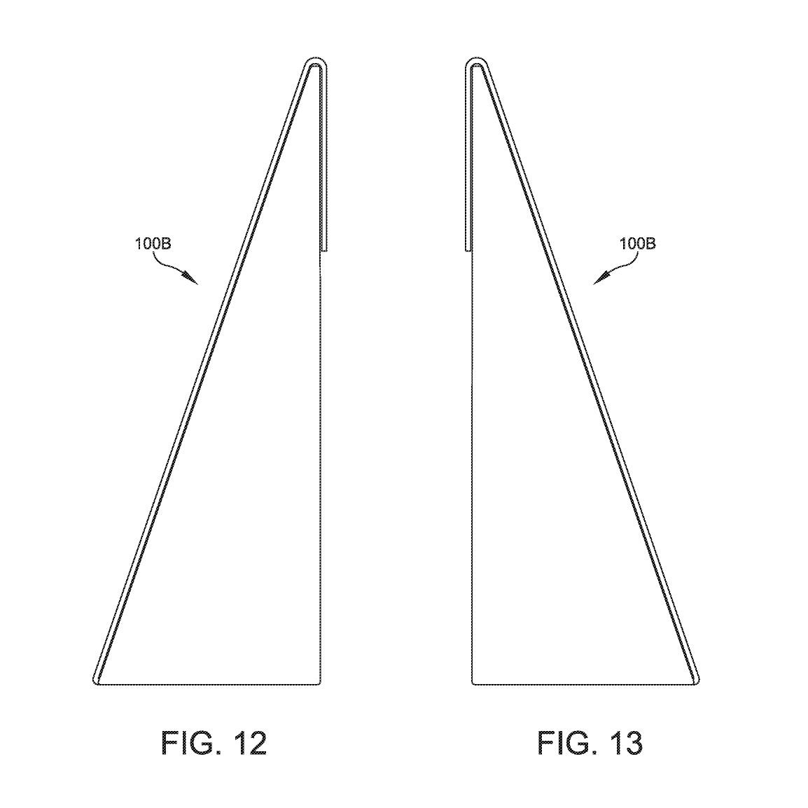

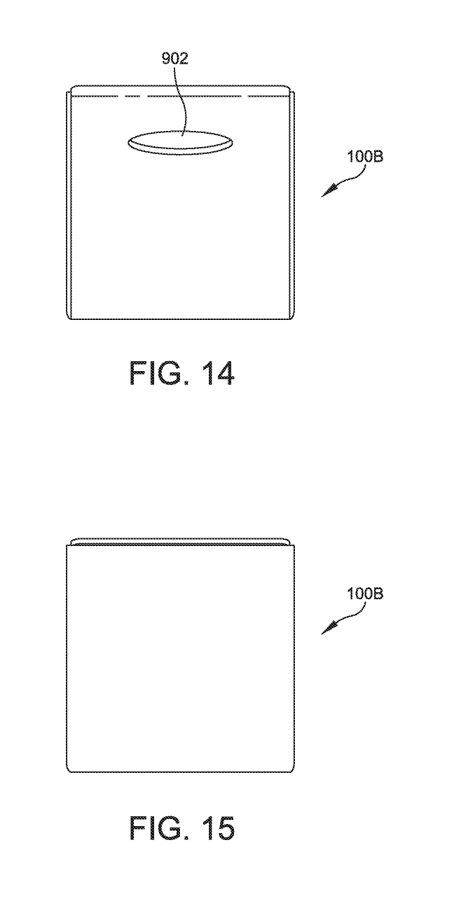

Referring now to FIGS. 9 through 16, this embodiment of the kit package 100B can be stood upright with its widened end 204 resting on a flat surface (not shown). Alternatively, the package kit may be mounted to a mounting rod or other similar structure (not shown) penetrating the circular mounting rod passage 902 penetrating the narrower upper section 904 of the package 100B. Mounting rod passage 902 can also be used to help carry the package 100B by inserting a finger through the passage 902.

The colorant mixing container and method may be different than as described above. For example, a wide variety of storage, injection, and mixing systems can be used to provide two compartments for the colorant and peroxide, respectively, and then mixing of the two. Exemplary such systems can include:
use of two syringe-like structures that can both suck in colorant and peroxide respectively and eject them into a mixing container of any type;
two-chamber syringe-like injectors, with mixing commenced at least by ejection of the colorant and peroxide through a single exit port; the chambers may be parallel to each other with each penetrated by a plunger, or the chambers may be laterally aligned and separated by a breakable seal, with one end of the two-chamber structure penetrated by a plunger and the other end having and ejection port;
two-camber plastic bags with a common exit port opposite a sealable opening end (such as by ziplock), with the colorant and peroxide stored separately, such as in separate ziplock bags, until injected into the two chambers, respectively; a plurality of such bags can be pre-formed in strip of plastic or other suitable material and the rolled up to be delivered to a user, so the user can tear off one two chamber bag as needed;
a two chamber ampule (with one chamber abutting the other laterally along the lateral length of the ampule), with each chamber having an injection port for receipt of colorant and peroxide, respectively, with the colorant and peroxide injectable through the ports in a variety of ways, one of which can be by separate ampules containing the colorant and peroxide respectively;
simply having two separate containers (of any suitable type, such as without limitation, toothpaste tube-like tubes with an ejection end opposite an openable and re-sealable end, re-sealable boxes, bottles, plastic containers, etc.) to store the colorant and peroxide separately prior to use by mixing them in any suitable other container or mixing bowl; and/or
a sealable box or oblong container, made of any suitable material, with a removable separator forming two laterally separated chambers in the container, one for colorant and one for peroxide.

With regard to application of colorant from the kit, the application tool could include: a sponge or other material for applying the colorant to hair; a plastic, rubber, metal, or silicon finger-tip cap with a resilient applicator material or brush on the external side of the cap, such as at the tip, side, or both; a blotter, or a bottle or container cap with an external applicator such as, for example, a brush, sponge, or blotter. In this regard, the kit can also include one or a pair of plastic or rubber gloves to prevent contact of the user's hand with the colorant and peroxide formulation.

With regard to the removable seals used in containers such as specified supra, the seals can have other features, such as a pin hole for passage of air through the seal. When placed in position adjacent colorant or peroxide in the container, a sealing adhesive or tape can be used to seal the hole, or in the case of a seal adjacent colorant, the colorant can penetrate the hole and seal it.

The seals may be secured in position in a container in differing ways, such as by mating channels in the interior wall of the container or by threads in the periphery of the seal for threading of the seal into mating threads in the container interior wall. Alternatively or in addition, the sealing structure can be provided by a plug structure with an O-ring surrounding the periphery of the plug. Further, a given seal (external or internal) may be puncturable so that, rather than removing a seal to accomplish mixing, the seal is left in place and punctured by a tool to allow colorant to then be mixed with peroxide in the container.

With regard to the top side seal or other seal covering a passage into a container structure, the sealing function can be accomplished in other ways. Other such structures can include plugs, caps securable to mating structure on the container, such as by mating threads, resilient lip structure, etc.

With regard to mixing of colorant with peroxide, yet other mixing techniques can be used, including breaking or removal of a seal or other chamber-separating structure and simply shaking the container or using a rotatable mixing device penetrating a passage in the container. Mixing tools also can include a non-stick or hydrophobic surface, so that colorant or a colorant component will be less likely to stick to that surface.

With regard to indicia for identifying the quantity of material placed into a chamber, container, etc., this indicia can be provided in the structures identified herein in any other suitable ways. The container, bag, chamber structure, etc., can be transparent or sufficiently translucent and have the indicia marked on the outside of structure.

The various alternative structures and methods described above may be mixed and matched as desired. Features may also be deleted as desired. Generally speaking, however, one objective of the method is to prevent colorant from oxidizing excessively prior to its use to color hair or otherwise be utilized to accomplish coloration.

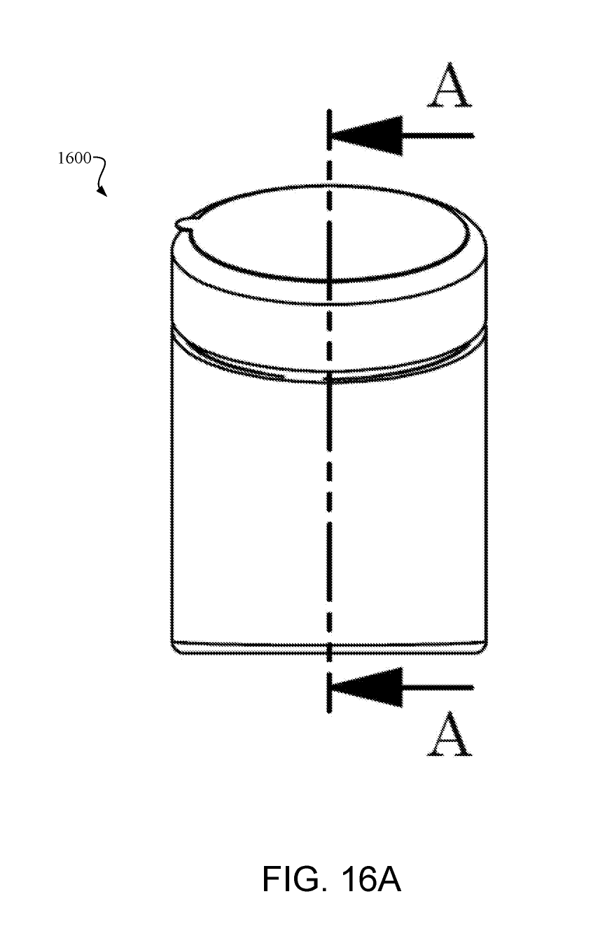

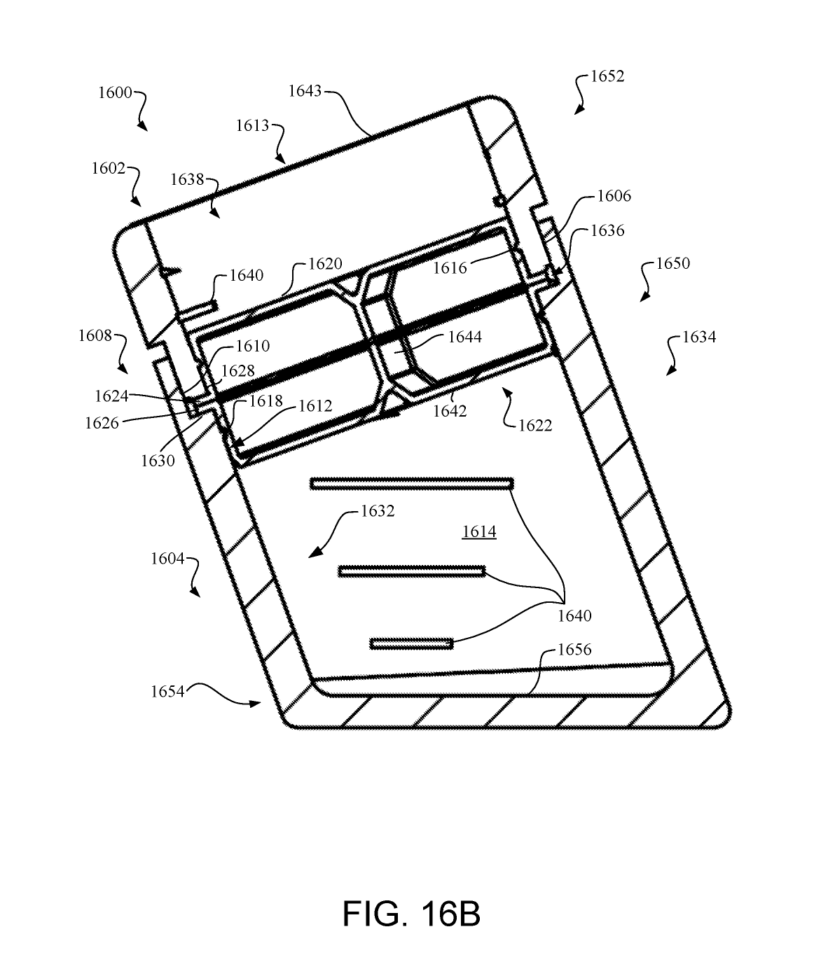

Referring now to FIGS. 16A and 16B and their depiction of an alternative example of a container 1600, the container 1600 includes a hair colorant container chamber or section 1602 and a peroxide container chamber or section 1604. As noted above, the differing container sections 1602, 1604 can contain materials other than, or in addition to, hair colorant and peroxide.

The container 1600 may include a hair colorant container chamber 1602 removably mountable to a peroxide container chamber 1604, a first interior seal 1620 threadably engageable with an interior first seal surface 1611 within the hair colorant container chamber 1602, and a second interior seal 1622 threadably engageable with an interior second seal surface 1612 within the peroxide container chamber 1604. In some cases, the hair colorant container chamber 1602 is threadably mountable to the peroxide container chamber 1604.

The hair colorant container chamber 1602 and peroxide container chamber 1604 each have a generally tubular inner periphery 1632 and a tubular outer periphery 1634. The container 1600 may also include a third removable seal 1613 removably mountable on the hair colorant container chamber 1602 spaced from the first interior seal 1620 or on the peroxide container chamber 1604 spaced from the second interior seal 1622

The first interior seal 1620 and second interior seal 1622 may each have a tubular seal body section 1627 and an annular sealing flange 1624, 1626 extending radially outwardly from the tubular seal body section 1627. Each annular sealing flange 1624, 1626 may be mountable to abut a sealing flange slot 1636 cooperatively providable by the hair colorant container chamber 1602 and peroxide container chamber 1604. The third removable seal 1613 may have a disk body 1643 secured to a mixing passage 1638 penetrating the hair colorant container chamber 1602 or the peroxide container chamber 1604.

The first interior seal 1620 and second interior seal 1622 may each have a central seal body 1627 and a finger grip tab 1644 extending from the central seal body 1627. The first interior seal 1620 and second interior seal 1622 may each have a sealing disk section 1642 extending across the tubular body section 1627 and the finger grip tab 1644 extends from sealing disk 1642. The first interior seal 1620 and the second interior seal 1622 may each consist essentially of the same integral unitary structure.

The hair colorant container section 1602 may have a first seal fill level indicator 1640 on the first seal interior surface 1611 and the peroxide container section 1604 has a second seal fill level indicator 1640 on the second seal interior surface 1612. The fill level indicator 1640 may be a notch, groove, recess, protrusions or another type of marking incorporated into the interior surface 1611 of either or both of the container sections 1602, 1604. These fill level indicators 1640 may provide a visual marking that allows the user to gauge the amount of liquid or other type of material that is added to each compartment the container sections 1602, 1604. For example, the user may fill the fill level indicator 1640 to understand how much liquid or other type of material should be added into the container sections 1602, 1604. A method of using the container 1600 may include filling each or both of the container sections 1602, 1604 to at least one of the fill level indicators 1640 incorporated on the interior surface 1611 of either of the container sections 1602, 1604 before closing off the container sections 1602, 1604 with their respective interior seals 1620, 1622.

The fill level indicator 1640 of the hair colorant container section 1602 may be generally parallel with the third removable seal 1613. When the hair colorant container section 1602 is receiving the hair colorant liquid or other type liquid, the hair colorant container section 1602 may be oriented so that the third removable seal 1613 forms the base end of the container section 1602 while the compartment of the hair colorant container section 1602 is accessible through the open, narrower end 1608. As liquid or another type of material is added to the hair colorant container section 1602 through the narrower end 1608, the liquid may settle so that the liquid's level is substantially parallel with the third removable seal 1613 and the fill level indicator 1640. With the hair colorant container section 1602 filled, the first interior seal 1620 may be secured within the interior surface 1611 of the hair colorant container section 1602 to close off the narrower end 1608 of the hair colorant container section 1602 and to seal the hair colorant or other type of material within the hair colorant container section 1602.

The fill level indicator 1640 of the peroxide container section 1604 may be generally parallel with the base end 1656 of the peroxide container section 1604. When the peroxide container section 1604 is receiving the peroxide or other type liquid, the peroxide container section 1604 may be oriented so that compartment of the peroxide container section 1604 is accessible through the open, upper end 1656. As the peroxide or another type of material is added to the peroxide container section 1604 through the upper end 1650, the peroxide may settle so that the liquid's level is substantially parallel with the base end 1654 of the peroxide container section 1604 and the fill level indicator 1640. With the peroxide container section 1604 filled, the second interior seal 1622 may be secured within the interior surface 1612 of the peroxide container section 1604 to close off the upper end 1650 of the peroxide container section 1604 and to seal the peroxide or other type of material within the peroxide container section 1604.

The hair colorant container section 1602 includes an external thread 1606 located proximate a lower, narrower end 1608 of the hair colorant container section 1602. The peroxide container section 1604 includes a first internal thread 1610 sized and spaced to threadably engage the external thread 1606 of the hair colorant container section 1602.

With the hair colorant container section 1602 and the peroxide container section 1604 threaded together, the hair colorant container section 1602 and the peroxide container section 1604 collectively form a single container 1600. The single container 1600 includes an interior periphery 1612 defining a cavity 1614 cooperatively provided by both the hair colorant container section 1602 and the peroxide container section 1604. The cavity 1614 may be divided into compartments located in the hair colorant container section 1602 and the peroxide container section 1604 by the first and second interior seals 1620, 1622.

The seal surface 1611 of the hair colorant container section 1602 includes an internal colorant seal thread 1616, and the interior periphery 1612 of the peroxide container section 1602 includes an internal peroxide seal thread 1618. The first interior seal 1620 has external first seal threads 1621 that matingly thread to the internal colorant seal thread 1616 of the hair colorant container section 1602, and a second interior seal 1622 has external second seal threads 1623 that matingly thread to the internal colorant seal thread 1618 of the peroxide container section 1604.

The first interior seal 1620 includes a first annular flange 1624 extending transversely, outwardly away from the annular central body section 1627 of the first interior seal 1620, and the second interior seal 1620 includes a second annular flange 1626 extending transversely, outwardly away from the annular central seal body section 1627 of the second interior seal 1622. The first annular flange 1624 sealingly abuts a circular first section shoulder 1628 in the narrower, open end 1608 of the hair colorant container section 1602, and the second annular flange 1626 sealingly abuts a circularly internal shoulder 1630 of the upper, open end 1650 of the peroxide container section 1604. As the hair colorant container section 1602 and the peroxide container section 1604 are threaded together, the shoulders 1628, 1630 move closer together forcing the first and second annular flanges 1624, 1626 to sealingly abut each other. In some examples, the hair colorant container section 1602 and the peroxide container section 1604 are threaded tightly enough together that the first and second annular flanges 1624, 1626 are compressed against each other. In some cases, a gap or slot 1636 between the shoulders 1628, 1630 is formed and the first and second annular flanges 1624, 1626 reside in the slot 1636 under compression by the shoulders 1628, 1630.

The first and second interior seals 1620, 1622 may prevent leaking of the respective gases, liquids, and/or solid materials from their respective container sections 1602, 1604 at the interface between the external threads 1632 of the interior seals and the internal threads 1616, 1618 of the container sections' interior peripheries 1612. In some situations, leaking is further prevented by compression of the first annular flange 1624, the second annular flange 1626, or both. In some cases, the leaks are prevented or at least minimized collectively by both the compression of at least one of the annular seal flanges 1624, 1626 and the interface of at least one of the interior seals 1620, 1622 and the container's inside surface 1632.

The hair colorant container section 1602 has a central generally tubular body 1627 with a narrower, lower end 1608 extending from a wider upper end 1652. The hair colorant container section's threads 1616 extends outwardly from the interior of the narrower, lower end 1608 to threadably interlock with the mating opposed threads 1616 in the first interior seal 1620, which is oriented in the hair colorant container section 1602 with the finger grip tab 1644 extending downwardly from the disk section 1642 of the first interior seal 1620.

The peroxide container section 1604 also has a generally tubular central section 1627. The tubular section 1627 terminates in a solid lower base 1654 spanning across the lower end 1656 of the tubular section 1627 at 5 to 45 degree angle to the central axis of the peroxide container section's generally tubular section 1627. The upper end 1650 of this tubular section 1627 terminates in a narrower end 1608 at the hair colorant container section 1602 when the container sections are threadedly connected.

The peroxide container section 1604 has a thinner upper end 1650 extending from a thicker lower end 1654. Threads 1606 extending radially inwardly from the upper thinner section 1650 threadably mate and interlock with threads 1610 extending radially outwardly from the narrow end 1608 of the hair colorant container section 1602.

The narrower end 1608 of the hair colorant section 1602 is spaced from the radially inwardly extending upper circular end 1650 of the peroxide container section 1604. The narrower end 1608 of the hair colorant container section 1602 and the upper circular end 1650 of the peroxide container section 1604 cooperatively provide a sealing flange slot or cavity 1636. When the hair colorant container section 1602 and the peroxide container section 1604 are threadedly connected, the shoulder 1628 of the hair colorant section 1602 and the shoulder 1630 of the peroxide container section 1604 may compress the flanges 1624, 1626 of the interior seals 1620, 1622 to provide barrier that prevents fluids or gases from escaping from the cavity 1614 of the container. In some cases, the compressed flanges 1624, 1626 form the primary seal to prevent the leakage. In other examples, the compressed flanges 1624, 1626 form a back-up or secondary barrier to prevent such a leakage.

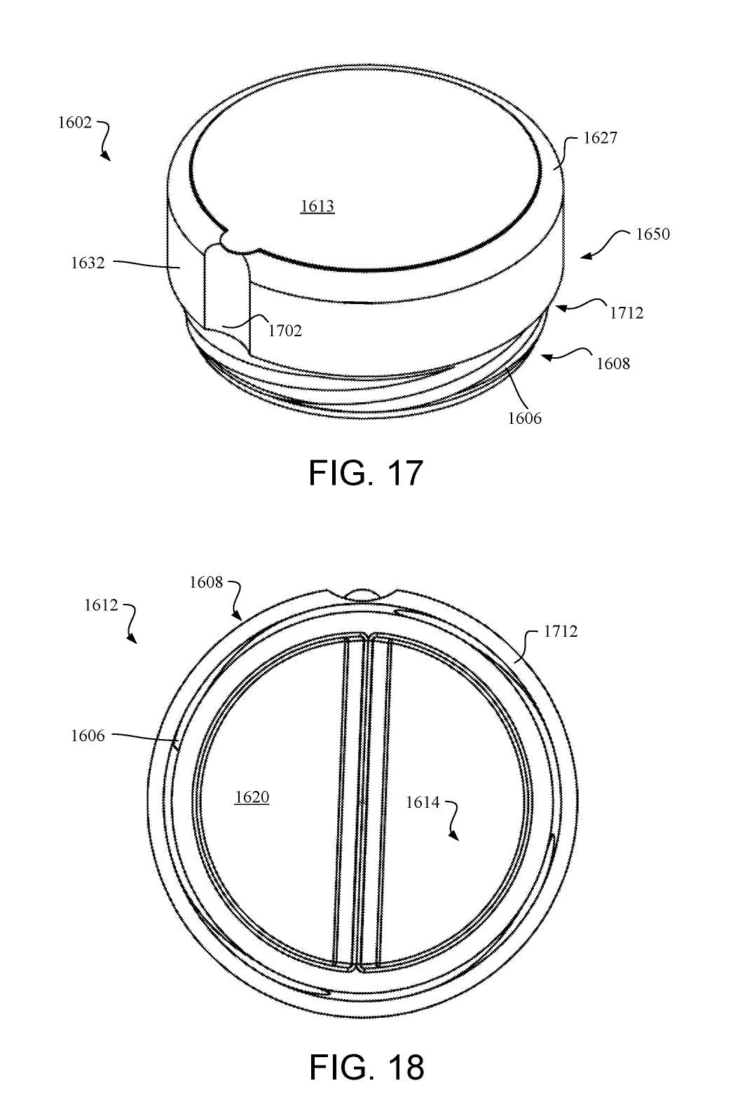

FIGS. 17-21 depict various views of the hair colorant container section 1602. The outside surface 1632 of the hair colorant container section 1602 is generally circular with an indent 1702 for steadying the hair colorant container section 1602 when a user opens or closes the container by rotating the hair colorant container section 1602 relative to the peroxide container section 1604. An opening (not shown) is defined in the top of the hair colorant container section 1602, which is covered by the third removable seal 1613. The generally circular body 1627 of the hair colorant container section 1602 has a first, wider end 1650 and a second, narrower end 1608 that is narrower than the wider end 1650. The transition between the wider end 1650 and the narrower end 1608 forms an outside shoulder 1712. The external container thread 1606 is formed in the narrower end 1608 of the hair colorant container section 1602.

FIG. 18 depicts a bottom plan view of the hair colorant container section 1602. In this view, the first interior seal 1620 is positioned within the cavity 1614 of the hair colorant container section 1602. The first interior seal 1620 includes an annular flange 1624 that covers the shoulder 1628 of the narrower end 1608 of the hair colorant container section 1602. The external container thread 1606 of the second diameter 1710 is depicted in front of the outside shoulder 1712.

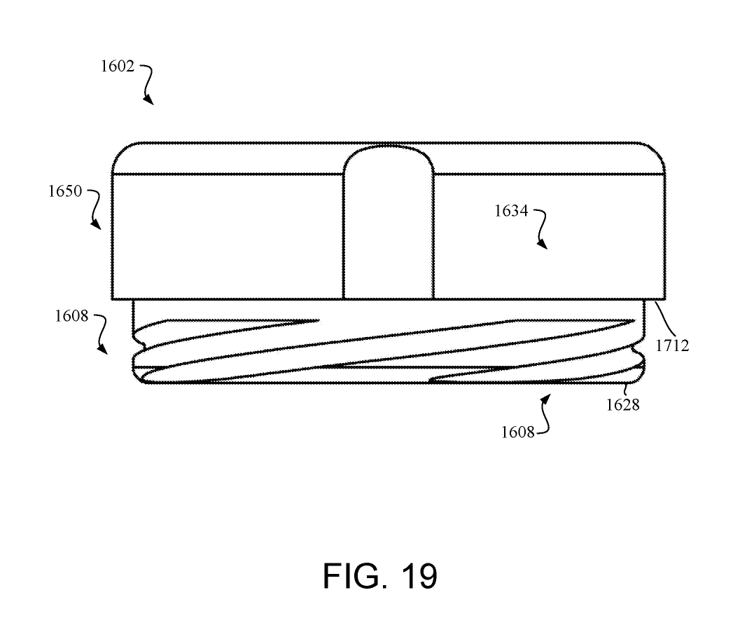

FIG. 19 depicts a side plan view of the hair colorant container section 1602 without the first interior seal 1620. In this example, the outside shoulder 1712 connects the wider end 1650 to the narrower end 1608. The shoulder 1628 on the narrower end 1608 is generally parallel with the outside shoulder 1712. The external container thread 1606 is depicted on the narrower end 1608. The external container thread 1606 includes a first thread start 1720 and a second thread start 1722 depicted in FIG. 19. While not shown in this illustration, in some embodiments, the far side of the outside surface of the second diameter may include additional thread starts. In some examples, the external container thread may include four thread starts. The thread pitch may form an angle between 15 and 45 degrees with the surface of the shoulder 1628 on the narrower end 1608 of the hair colorant container section 1602.

The narrower end 1608 of the hair colorant container section 1602 protrudes below the outside shoulder 1712. Proximate the narrower end 1608 of the hair colorant container section 1602, the outside surface 1634 includes the external container thread 1606 and the seal surface 1611 includes the internal seal thread 1616. In the depicted example, the external container thread 1616 includes a thread width that is at least 50% larger than the thread width of the internal seal thread 1616. In some examples, the external container thread width is between 25% and 300% wider than the internal seal thread 1616.

The annular flange 1624 of the first interior seal 1620 extends outwardly away from the body 1627 of the interior seal 1620 across the face of the external shoulder 1628 of the narrower end 1608 of the hair colorant container section 1602. As the hair colorant container section 1602 is threadedly engaged with the peroxide container section 1604, the annular flange 1624 prevents the exterior shoulder 1628 from coming into direct contact with the inside shoulder 1630 of the peroxide container section 1604.

In some cases, the hair colorant container section 1602 may be removed from the peroxide container section 1604 and turned upside down where the third removable seal 1613 becomes a base of the hair colorant container section 1602. In this situation, a liquid in the hair colorant container section 1602 may level off to be substantially parallel with the third removable seal 1613 rather than being leveled off at an angle caused by the angled based end 1646 of the peroxide container section 1604 when the container sections 1602, 1604 were threaded together. These hair colorant indicators 1640 of the hair colorant container section 1602 may also be used as fill lines when the hair colorant is initially added to the cavity 1614 of the hair colorant container section 1602. After filling the cavity 1614, the interior seal 1620 may be threaded into place to close the cavity 1614 and prevent leaks.

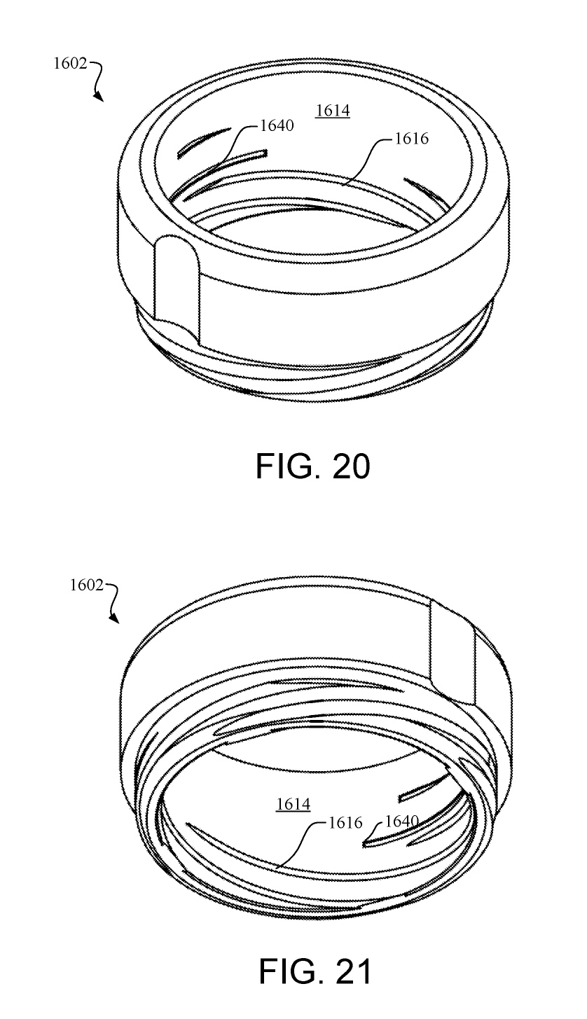

FIGS. 20 and 21 depict top and bottom views of the hair colorant container section 1602 without the interior seal 1620 or the third removable seal 1613 disposed therein. As can be seen depicted in these views, the hair colorant container section 1602 has a generally annular body 1627. The inside seal surface 1614 includes the hair colorant indicators 1640 and the internal seal thread 1616 on the same diameter. The third removable seal 1613 may close off the wider end 1650 of the hair colorant container section 1602, and the first interior seal 1620 may close off the narrower end 1608 of the hair colorant container section 1602 to form a compartment that is suitable for holding at least one of a gas, a liquid, a powder, another type of solid, another type of material, or combinations thereof.

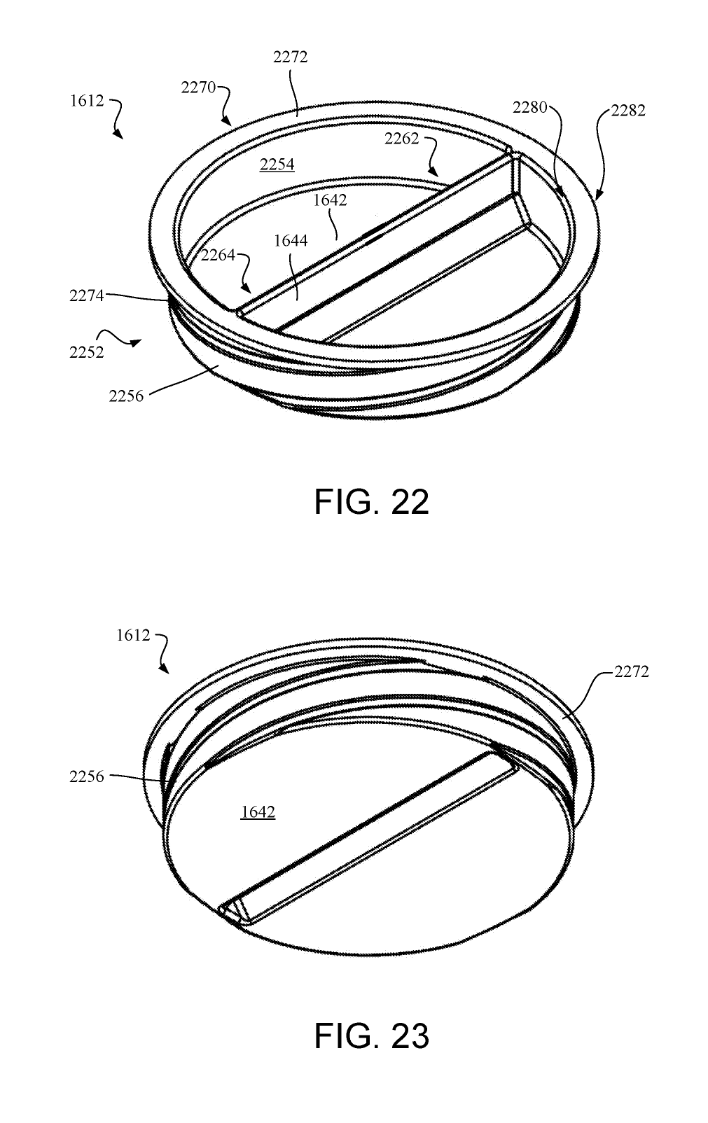

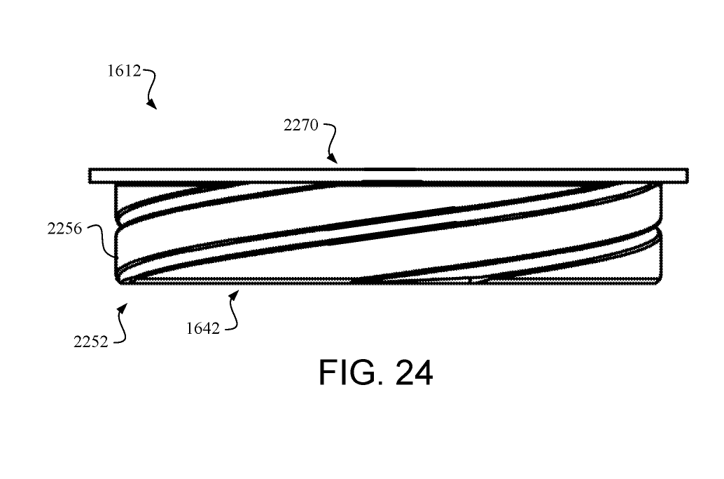

FIGS. 22-24 depict various views of an interior seal 2250 that engages with the inside internal thread 1616, 1618 of either the hair colorant container section 1602 or the peroxide container section 1604. Each of the two interior seals 1620, 1622 have a generally central solid disk section 1642 with a generally tubular or annular section 2252 extending transversely from an outer circumferential side 2256 of the disk section 1642. The seal threads 2274 penetrate the outer peripheral side of the tubular section 1627. A finger grip wall or tab 1644 extends (i) transversely from the side of the disk 1642 surrounded by the tubular section 2252 across the diameter of the disk 1642 and (ii) a transversely from opposed interior sides 2254 of the tubular section 2252. The annular sealing flange 2272 extends transversally outwardly from the outer periphery 2256 of the open end 2272 of the tubular section 2252 opposite the latter's closed end formed by the disk section 1642 and its intersection with the tubular section 2252. The seal 1612 may be molded to provide a single, integral structure.

The sealing flanges 2272 of the opposed seals 1620, 1622 penetrate the sealing flange cavity slot 1636. The open end 2270 of the upper seal's sealing flange 2272 abuts the shoulder 1628 of the narrower end 1608 of the hair colorant container section 1602, and the open end 2270 of the lower seal's sealing flange 2272 sealingly abuts the shoulder 1630 of the upper end 1650 of the peroxide container section 1604. The open end 2270 of the upper seal's finger grip wall 1644, opposite the wall's junction with the central disk section 1642 of the upper seal 1620, abuts the opposed open end 2270 of the lower seal's finger grip wall 1644.

In FIG. 22, the interior seal 1612 includes an annular body 2252 with a seal inside surface 2254 and a seal outside surface 2256. A finger grip 1644 is connected to the seal inside surface 2254 such that a user can orient the interior seal 1612 by moving the finger grip 1644. In this example, a first side 2262 of the finger grip 1644 is connected to the inside surface, a body of the finger grip 1644 spans across the cavity 1614 of interior seal 1612, and a second side 2264 of the finger grip 1644 that also connects to another portion of the seal's inside surface 2254.

The disk section 1642 of the annular body 2252 closes off the cavity 1614 of the annular body 2252 preventing the passage of gases, liquids, solids, and/or other types of materials. In some cases, the disk section 1642 is connected to the finger grip 1644. On an open side 2270, which is opposite the disk section 1642, an annular flange 2272 extends outwardly away from the annular body 2252. The annular flange 2272 has an inside diameter 2280 that is approximately the same as the inside surface 2254 of the annular body 2252. The outside diameter 2282 of the annular flange 2272 has a greater width than the outside diameter 2256 of the annular body 2252.

FIG. 23 depicts that the annular flange 2272 overhangs the outside diameter 2256 of the annular body 2252. In this example, the annular flange 2272 protrudes farther beyond the distance away from the annular body 2252 than the external seal thread 2274 of the seal outside surface 2256.

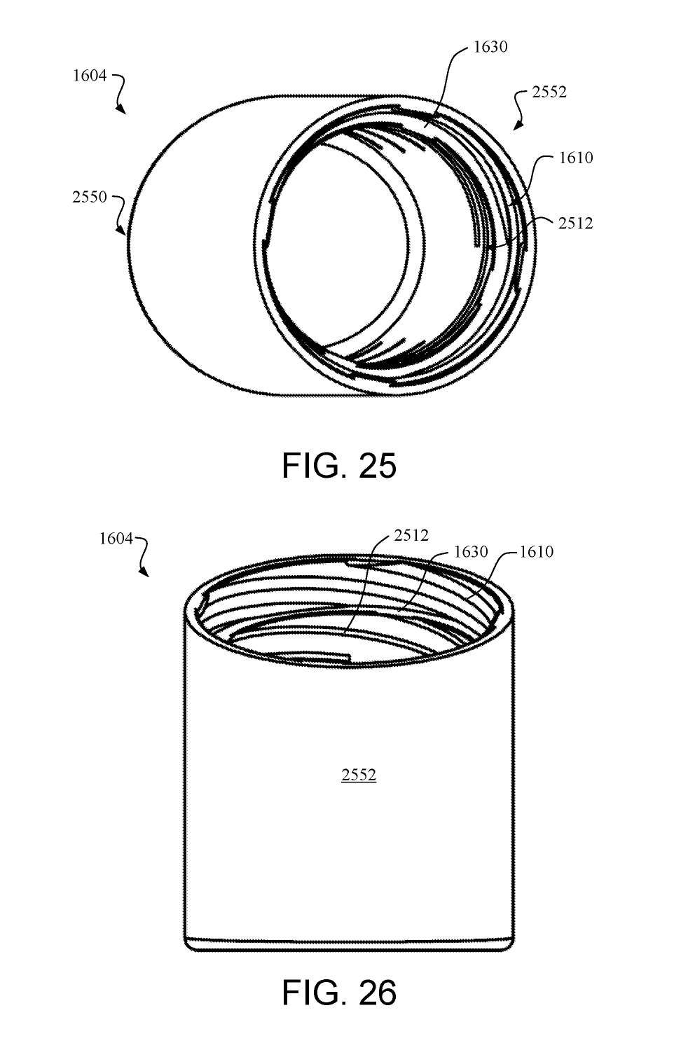

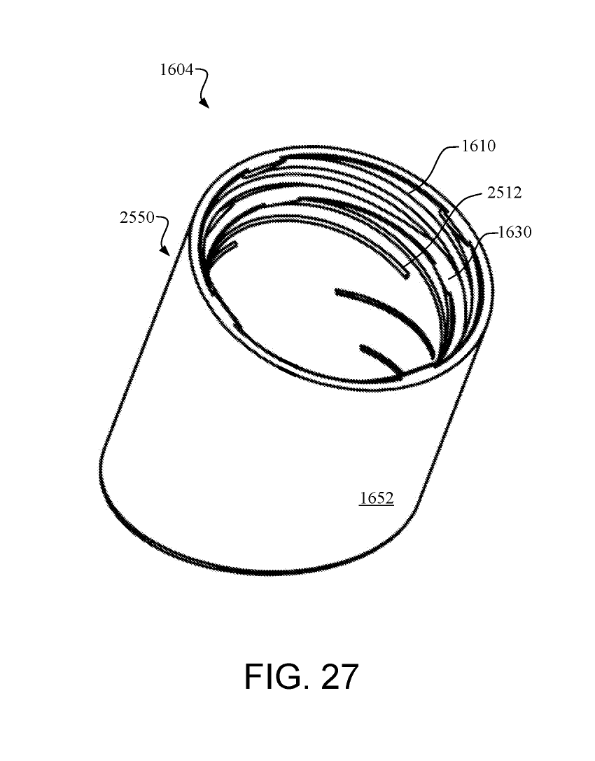

FIGS. 25-27 depict top and side views of the peroxide container section 1604. In these examples, the internal container thread 1610 has a steeper thread pitch than the internal seal thread 2512. Additionally, a first side 2550 of the peroxide container section 1604 is longer than a second side 1652 that is opposite the first side 2550. In this example, the inside shoulder 1630 is substantially perpendicular to a central axis of the peroxide container section 1604. Thus, with the peroxide container section 1604 having one side longer than the other, the inside shoulder 1630 is not substantially parallel with a base of the peroxide container section 1604. Rather, the inside shoulder 1630 is angled by at least 15 degrees to 75 degrees with respect to the base of the peroxide container section 1604.

The annular flange of the second interior seal 1622 may cover at least a portion of the internal shoulder 1630 of the peroxide container section 1604 when the second interior seal 1622 is engaged with the peroxide container section 1604. In some embodiments, the annular flange 2272 of the first interior seal 1620 and the annular flange 2272 of the second interior seal 1622 may be disposed in the slot 1636 between narrower end 1608 of the hair colorant container section 1602 and the internal shoulder 1630 of the peroxide container section 1604 when the hair colorant container section 1602 and the peroxide container sections 1604 are threaded together. In some cases, the annular flange 2272 of the first interior seal 1602 and the annular flange 2272 of the second interior seal 1604 may be pressed together by the shoulder 1628 of the narrower end 1608 of the hair colorant container section 1602 and the internal shoulder 1630 of the peroxide container section 1604 when the hair colorant container section 1620 and the peroxide container section 1604 are threaded together. In some situations, the seal thread pitch of the internal seal thread 2274 engaged with the second interior seal 1622 is steeper than the internal body thread 1612 engaged with the exterior body thread 1606 of the hair colorant container section 1602. In some cases, the internal seal thread 1618 engaged with the second interior seal 1622 is steeper and has more thread starts than the body internal thread 1616 engaged with the exterior body thread 1606 of the hair colorant container section 1604.

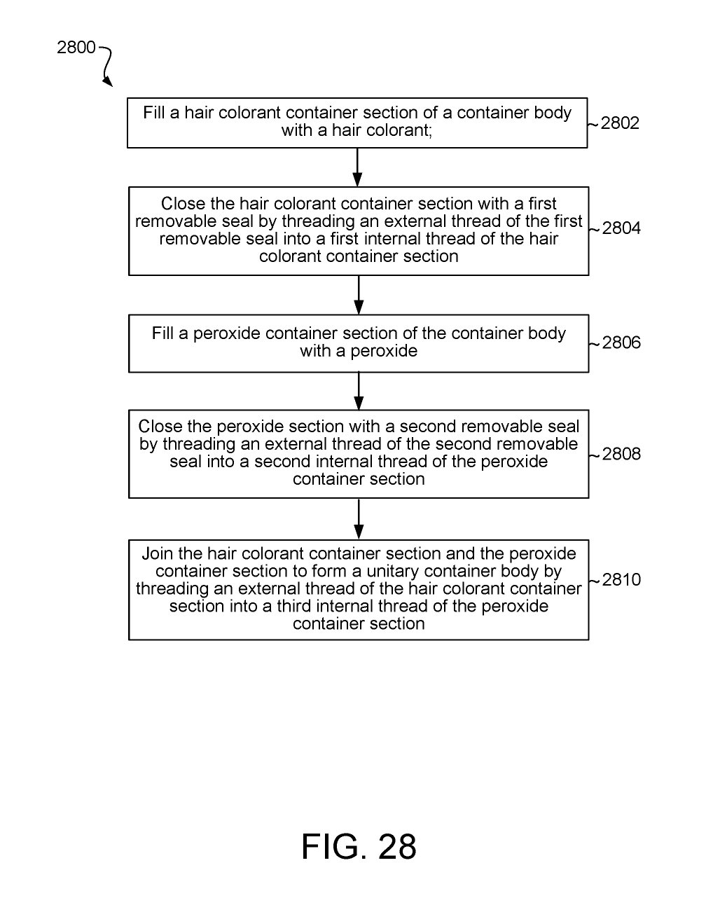

FIG. 28 depicts an example of a method 2800 of using a container. In this example, the method includes filling 2802 a hair colorant container section of a container body with a hair colorant kit portion, threading 2804 an external first seal thread of a first interior seal into an internal hair colorant section thread of the hair colorant container section, filling 2806 a peroxide container section of the container body with a peroxide, threading 2808 an external second seal thread of a second interior seal to an internal peroxide section thread of the peroxide container section, and threading 2810 a second hair colorant section thread of the hair colorant container section to a second peroxide section thread of the peroxide container section. In some cases, the method may also include custom development of a hair colorant, applying an application portion of the hair colorant to hair, and utilizing an unapplied portion of the hair colorant as the hair colorant kit portion.

### Additional Considerations:

Articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y).

The term “and/or” shall also be interpreted to be inclusive (e.g., “x and/or y” means one or both x or y). In situations where “and/or” or “or” are used as a conjunction for a group of three or more items, the group should be interpreted to include one item alone, all the items together, or any combination or number of the items. Moreover, terms used in the specification and claims such as have, having, include, and including should be construed to be synonymous with the terms comprise and comprising.

Unless otherwise indicated, all numbers or expressions, such as those expressing dimensions, physical characteristics, and the like, used in the specification (other than the claims) are understood to be modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

All disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

All disclosed numerical values are to be understood as being variable from 0-100B % in either direction and thus provide support for claims that recite such values or any and all ranges or subranges that can be formed by such values. For example, a stated numerical value of 8 should be understood to vary from 0 to 16 (100B % in either direction) and provide support for claims that recite the range itself (e.g., 0 to 16), any subrange within the range (e.g., 2 to 12.5) or any individual value within that range (e.g., 15.2).

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used in this document shall mean” or similar language (e.g., “this term means,” “this term is defined as,” “for the purposes of this disclosure this term shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained in this document should be considered a disclaimer or disavowal of claim scope.

The subject matter recited in the claims is not coextensive with and should not be interpreted to be coextensive with any embodiment, feature, or combination of features described or illustrated in this document. This is true even if only a single embodiment of the feature or combination of features is illustrated and described in this document.

What is claimed is:

1. A hair coloring container apparatus comprising in combination:
a hair colorant container removably mountable to a peroxide container;
a first interior seal threadably engageable with an interior first seal surface within the hair colorant container; and
a second interior seal threadably engageable with an interior second seal surface within the peroxide container.

2. The hair coloring container apparatus of claim 1 wherein the hair colorant container is threadably mountable to the peroxide container.

3. The hair coloring container apparatus of claim 1 wherein the hair colorant container and peroxide container each have a tubular outer periphery.

4. The hair coloring container apparatus of claim 2 wherein the hair colorant container and peroxide container each have a tubular outer periphery.

5. The hair coloring container apparatus of claim 1 further comprising a third removable seal removably mountable on (i) the hair colorant container spaced from the first interior seal or (ii) the peroxide container spaced from the second interior seal.

6. The hair coloring container apparatus of claim 4 further comprising a third removable seal removably mountable on the hair colorant container spaced from the first interior seal or the peroxide container spaced from the second interior seal.

7. The hair coloring container apparatus of claim 1 wherein the first interior seal and second interior seal each have a tubular seal body section and an annular sealing flange extending radially outwardly from the tubular seal body section.

8. The hair coloring container apparatus of claim 6 wherein the first interior seal and second interior seal each have a tubular seal body section and an annular sealing flange extending radially outwardly from the tubular seal body section.

9. The hair coloring container apparatus of claim 7 wherein each annular sealing flange is mountable to abut a sealing flange slot cooperatively providable by the hair colorant container and peroxide container.

10. The hair coloring container apparatus of claim 8 wherein each annular sealing flange is mountable to abut a sealing flange slot cooperatively providable by the hair colorant container and peroxide container.

11. The hair coloring container apparatus of claim 5 wherein the third removable seal has a disk body secured to a mixing passage penetrating the hair colorant container or the peroxide container.

12. The hair coloring container apparatus of claim 8 further comprising a third removable seal having a disk body removably mounted on (i) the hair colorant container spaced from the first interior seal or (ii) the peroxide container spaced from the second interior seal.

13. The hair coloring container apparatus of claim 1 wherein the first interior seal and second interior seal each have a central seal body and finger grip tab extending from the central seal body.

14. The hair coloring container apparatus of claim 12 wherein the first interior seal and second interior seal each have a sealing disk section extending across the tubular body section and a finger grip tab extending from sealing disk.

15. The hair coloring apparatus of claim 1 wherein the first removable seal and the second interior seal each consist essentially of the same integral unitary structure.

16. The hair coloring apparatus of claim 14 wherein the first removable seal and the second interior seal each consist essentially of the same integral unitary structure.

17. The hair coloring apparatus of claim 1 wherein the first removable seal has a first seal fill level indicator on the first seal interior surface and the second interior seal has a second seal fill level indicator on the second seal interior surface.

18. The hair coloring apparatus of claim 14 wherein the first removable seal has a first seal fill level indicator on the first seal interior surface and the second interior seal has a second seal fill level indicator on the second seal interior surface.

19. A method of containing hair color retouching constituents, comprising:
A. filling a hair colorant container section of a container body with a hair colorant kit portion;
B. threading an external first seal thread of a first interior seal into an internal hair colorant section thread of the hair colorant container section;
C. filling a peroxide container section of the container body with a peroxide;
D. threading an external second seal thread of a second interior seal to an internal peroxide section thread of the peroxide container section;
E. threading a second hair colorant section thread of the hair colorant container section to a second peroxide section thread of the peroxide container section.

20. The method of containing hair color retouching constituents of claim 19 further comprising custom development of a hair colorant, applying an application portion of the hair colorant to hair, and utilizing an unapplied portion of the hair colorant as the hair colorant kit portion.
